(19) United States
(12) Patent Application Publication
(10) Pub. No.: US 2026/0347835 A1
(43) Pub. Date: Nov. 5, 2026

(54) ALLELE-SPECIFIC CRISPR gRNAS TARGETING MYH7 TO TREAT CARDIOMYOPATHY

(71) Applicant: The J. David Gladstone Institutes, a testamentary trust established under the Will of J. David, San Francisco, CA (US)

(72) Inventors: Philip H. Dierks, San Francisco, CA (US); Eric Alexander Simental, San Francisco, CA (US); Bruce Conklin, San Francisco, CA (US)

(21) Appl. No.: 19/655,336

(22) Filed: Apr. 22, 2026

Related U.S. Application Data
(60) Provisional application No. 63/793,526, filed on Apr. 23, 2025.

(51) Int. Cl. C12N 15/11; C12N 9/22; C12N 15/90

(52) U.S. Cl. CPC C12N 15/11; C12N 9/226; C12N 15/907; C12N 2310/20; C12N 2320/34

(57) ABSTRACT

Provided are guide RNAs, compositions, ribonucleoprotein complexes, expression systems, and methods for allele-specific editing of the MYH7 gene to treat cardiomyopathy. The guide RNAs target common single nucleotide polymorphisms (SNPs), including rs2069540, rs735711, rs7157716, rs2231126, and rs2069542, located in protein-coding exons of MYH7, rather than individual disease-causing mutations. CRISPR-Cas nuclease cleavage directed by the guide RNAs introduces genomic insertions or deletions that generate premature stop codons, instigating nonsense-mediated decay of the mutant allele transcript while preserving wild-type allele expression. This mutation-independent approach enables treatment of heterozygous dominant-negative MYH7 cardiomyopathy across a broad patient population using a single set of reagents.

### PRIORITY

This application claims the benefit of the filing date of U.S. provisional application Ser. No. 63/793,526, filed on Apr. 23, 2025, the disclosure of which is incorporated by reference herein.

### GOVERNMENT SUPPORT

This invention was made with government support under AG072052 awarded by the National Institutes of Health. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Apr. 15, 2026, is named 3730240US.xml and is 45,056 bytes in size.

### FIELD OF THE INVENTION

The present disclosure relates to guide RNA compositions and CRISPR-based gene editing methods for allele-specific silencing of a mutant MYH7 allele by targeting common heterozygous single nucleotide polymorphisms in protein-coding exons. The disclosure further relates to the use of such compositions and methods to treat dominant-negative cardiomyopathy associated with heterozygous MYH7 mutations.

### BACKGROUND

Cardiomyopathies are diseases of the heart muscle that affect the ability of the heart to pump blood. Inherited cardiomyopathies, including hypertrophic cardiomyopathy (HCM), dilated cardiomyopathy (DCM), and restrictive cardiomyopathy (RCM), represent a group of conditions with a genetic basis that collectively affect an estimated 1 in 200 to 1 in 500 individuals worldwide. Among the genes implicated in inherited cardiomyopathy, the MYH7 gene, which encodes the beta-myosin heavy chain (β-MHC) protein (also referred to as Myosin-7), is the second leading cause of genetic cardiomyopathy (de Frutos et al. 2022; Morita et al. 2008). The MYH7 gene is located on human chromosome 14 (23.41-23.44 Mb) and contains 40 exons.

The β-MHC protein is a component of the sarcomere, the contractile unit of cardiac muscle. Mutations in MYH7 alter the structure or function of the β-MHC protein, which in turn disrupts sarcomeric assembly or force generation in cardiomyocytes. Geisterfer-Lowrance et al. first identified the Arg403Gln mutation in MYH7 as a causative mutation for HCM (Geisterfer-Lowrance A A, et al. 1990. Cell. 62 (5): 999-1006). Since that initial identification, over 200 different mutations in MYH7 have been associated with cardiomyopathy. These mutations are estimated to account for approximately 40% of HCM cases and large percent of dilated cardiomyopathy (DCM as well). MYH7-cardiomyopathy is an autosomal-dominant disease in which a single copy of a variant MYH7 allele can cause cardiomyopathy. Different MYH7 mutations can cause enlargement of the left ventricle (HCM) as well as dilation of the left ventricle (DCM) representing different manifestations of the disease. MYH7-DCM is the most severe form of the disease often leading to heart transplantation. The most severe disease mutations can be present at birth, while less severe variants present later in life and may be influenced by changes in thyroid hormone function and/or physical stress.

MYH7 mutations associated with cardiomyopathy typically result in dominant-negative (DN) pathology. In a heterozygous individual carrying one wildtype MYH7 allele and one mutant MYH7 allele, both alleles are expressed, and the mutant β-MHC protein is incorporated into the sarcomere alongside the wild-type protein. The presence of the mutant protein disrupts normal sarcomeric function even in the presence of the wild-type protein. This dominant-negative effect persists as long as the mutant allele continues to be expressed. DN cardiomyopathy caused by MYH7 mutations is resistant to current pharmacologic therapies, and heart transplantation remains the only definitive treatment for patients who progress to heart failure.

The advent of genome editing technologies, including the CRISPR-Cas9 system, has made it technically possible to correct individual disease-causing mutations in the genome. However, the diversity of MYH7 mutations presents a practical barrier to mutation-specific approaches. With over 200 different mutations identified in MYH7 that cause cardiomyopathy, developing, optimizing, and obtaining regulatory approval for a separate set of CRISPR reagents (e.g., a guide RNA specific to each mutation) for each individual mutation would be impractical. Each mutation-specific guide RNA would require its own preclinical development and regulatory review.

### SUMMARY

With the advent of genome editing technologies such as CRISPR, correcting MYH7 mutations are technically possible, but since there are over 200 different mutations in the MYH7 gene resulting in cardiomyopathy, it is impractical to propose targeting each mutation one-by-one (e.g., each set of CRISPR reagents would require different approval by the FDA).

To meet this challenge, provided herein is a mutation-independent approach to MYH7-cardiomyopathy, that is referred to herein as allele-specific gene silencing. MYH7 is ideal for this approach, since in a disease state, only one allele carries the mutation, while the other allele carries the wild-type sequence. In addition, only one normal copy of MYH7 is sufficient for normal health (haplosufficient) in mice and humans (Pandya et al. 2006; Beecroft et al. 2019), suggesting that allele-specific inactivation of the disease allele will result in a phenotypically normal cardiomyocyte.

Provided herein are compositions, methods of editing, and methods of treating that address dominant-negative cardiomyopathy associated with mutations in the MYH7 gene. Rather than targeting individual disease-causing mutations, the compositions and methods described herein employ allele-specific guide RNAs that target common heterozygous single nucleotide polymorphisms (SNPs) located in protein-coding exons of MYH7, thereby enabling a mutation-independent approach to silencing a mutant MYH7 allele while preserving expression from a wild-type allele. Some embodiments provided methods and compositions to treat subjects with cardiomyopathy that have a heterozygous disease-causing MYH7 mutation on the same allele as the SNPs (rs2069540, rs735711, rs7157716, rs2231126 or rs2069542) targeted herein.

In one aspect, a composition is provided comprising a carrier, a guide RNA, and a Cas nuclease. The guide RNA comprises an RNA sequence having at least 95% sequence identity to a sequence selected from the group consisting of SEQ ID NOs: 1-6, 13-24 and 25. The guide RNA directs the Cas nuclease to a genomic site within a protein-coding exon of a human MYH7 gene comprising a heterozygous SNP. In some embodiments, the guide RNA comprises an RNA sequence selected from the group consisting of SEQ ID NOs: 14, 17, 18, 19, 22, 23, 24 and 25. In some embodiments, the Cas nuclease is selected from the group consisting of Streptococcus pyogenes Cas9 (SpCas9), Staphylococcus aureus Cas9 (SaCas9), Francisella novicida Cas12a (FnCas12a), Neisseria meningitidis Cas9 (Nme2Cas9), and combinations thereof. In some embodiments, the guide RNA and the Cas nuclease are complexed as a ribonucleoprotein (RNP). In some embodiments, the carrier comprises a lipid nanoparticle, a virus-like particle (VLP), or an adeno-associated virus (AAV) vector. In some embodiments, the composition further comprises a second guide RNA comprising an RNA sequence having at least 95% sequence identity to a sequence selected from the group consisting of SEQ ID NOs: 1-6, 13-24 and 25, wherein the second guide RNA targets a different SNP allele than the first guide RNA. In some embodiments, the composition comprises an expression vector comprising a first promoter operably linked to a nucleic acid encoding the guide RNA and a second promoter operably linked to a nucleic acid encoding the Cas nuclease. In some embodiments, at least one of the first promoter and the second promoter is a cardiac-specific promoter.

In another aspect, a method of editing an endogenous MYH7 gene in a cell is provided. The method comprises contacting the cell with a guide RNA comprising an RNA sequence having at least 95% sequence identity to a sequence selected from the group consisting of SEQ ID NOs: 1-6, 13-24 and 25, and a Cas nuclease. The guide RNA directs the Cas nuclease to cleave one allele of the MYH7 gene at a genomic site comprising a heterozygous SNP, thereby introducing an insertion or deletion that generates a premature termination codon in the cleaved allele. Expression of a mutant MYH7 protein encoded by the cleaved allele is reduced relative to expression prior to the contacting. In some embodiments, the premature termination codon instigates nonsense-mediated decay of an mRNA transcript from the cleaved allele. In some embodiments, the cell is heterozygous for a dominant mutation in the MYH7 gene, and the SNP is in cis with the dominant mutation on the same allele. In some embodiments, the guide RNA and the Cas nuclease are delivered to the cell as a ribonucleoprotein (RNP) complex. In some embodiments, the guide RNA and the Cas nuclease are delivered to the cell via a virus-like particle (VLP). In some embodiments, the guide RNA comprises an RNA sequence selected from the group consisting of SEQ ID NOs: 14, 17, 18, 19, 22, 23, 24 and 25. In some embodiments, the cell is an iPSC-derived cardiomyocyte or a cardiomyocyte. In some embodiments, the Cas nuclease is SpCas9 or an adenosine base editor (ABE) comprising a Cas9 nickase domain.

In a further aspect, a method of treating a subject having a heterozygous dominant mutation in the MYH7 gene correlated with cardiomyopathy is provided. The method comprises administering to the subject a guide RNA comprising an RNA sequence having at least 95% sequence identity to a sequence selected from the group consisting of SEQ ID NOs: 1-6, 13-24 and 25, and a Cas nuclease. The guide RNA directs the Cas nuclease to a genomic site within a protein-coding exon of the MYH7 gene comprising a heterozygous SNP. The SNP is in cis with the dominant mutation. The administering reduces expression of a mutant MYH7 protein from the allele carrying the dominant mutation in cardiac cells of the subject. In some embodiments, the guide RNA and the Cas nuclease are administered as a ribonucleoprotein complex within a virus-like particle (VLP) or a lipid nanoparticle (LNP). In some embodiments, the guide RNA comprises an RNA sequence selected from the group consisting of SEQ ID NOs: 14, 17, 18, 19, 22, 23, 24 and 25. In some embodiments, the administering comprises delivering the guide RNA and the Cas nuclease to cardiac tissue of the subject, and expression from a wild-type MYH7 allele in the cardiac tissue is maintained.

### DRAWINGS

The drawings illustrate generally, by way of example, but not by way of limitation, various embodiments discussed herein.

FIG. 1. Demonstration of Allele-specificity and editing efficiency of gRNAs (Spg022, Spg023, Spg008, Sp018) in iPS-derived cardiomyocytes. Allele-specificity and editing efficiency of guide RNAs targeting SNPs rs2069540 and rs735711 in iPSC-derived cardiomyocytes is shown. Data were generated through next-generation sequencing, and results are presented as percentage of total reads categorized as non-target unmodified, target unmodified, and modified.

The upper left panel shows results for Spg022, a variant-targeting guide directed to the alternate allele of rs2069540. Untransfected cells show approximately equal proportions of non-target unmodified reads (blue) and target unmodified reads (red), with no modified reads detected. Upon transfection with Spg022, modified reads (gray) appear, representing editing of the target allele, while the non-target allele remains largely unmodified.

The upper right panel shows results for Spg023, a reference-targeting guide directed to the reference allele of rs2069540. In untransfected cells, non-target unmodified and target unmodified reads are present in approximately equal proportions. Upon transfection with Spg023, modified reads appear at the target (reference) allele, while the non-target (alternate) allele remains largely unmodified, demonstrating allele-specificity in the opposite direction relative to Spg022.

The lower left panel shows results for Spg008, a variant-targeting guide directed to the alternate allele of rs735711. Untransfected cells show approximately equal proportions of non-target unmodified and target unmodified reads. Upon transfection with Spg008, modified reads appear at the target allele, with the non-target allele remaining largely unmodified.

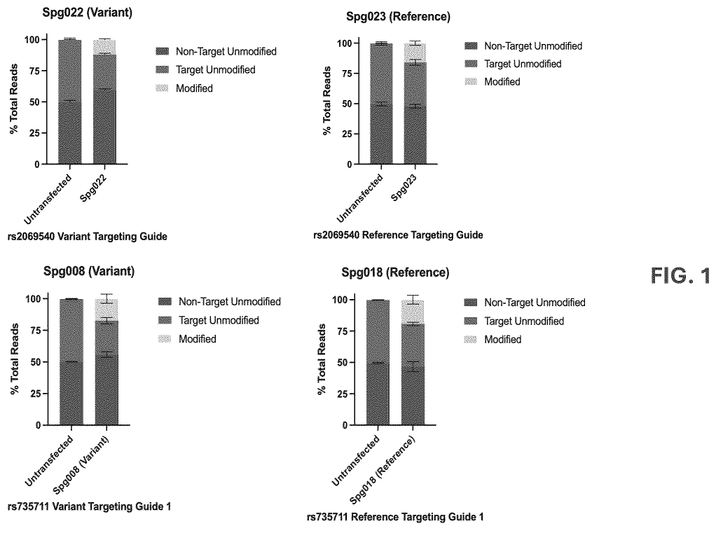

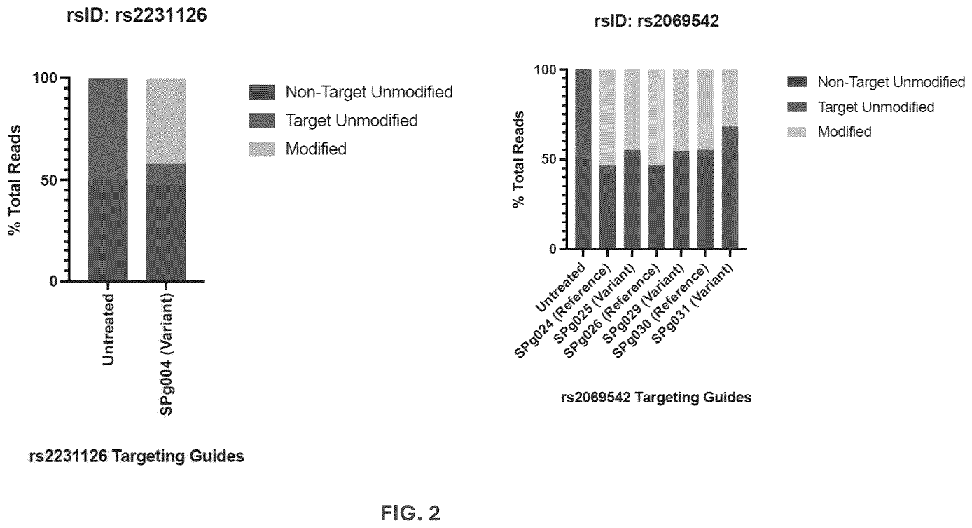

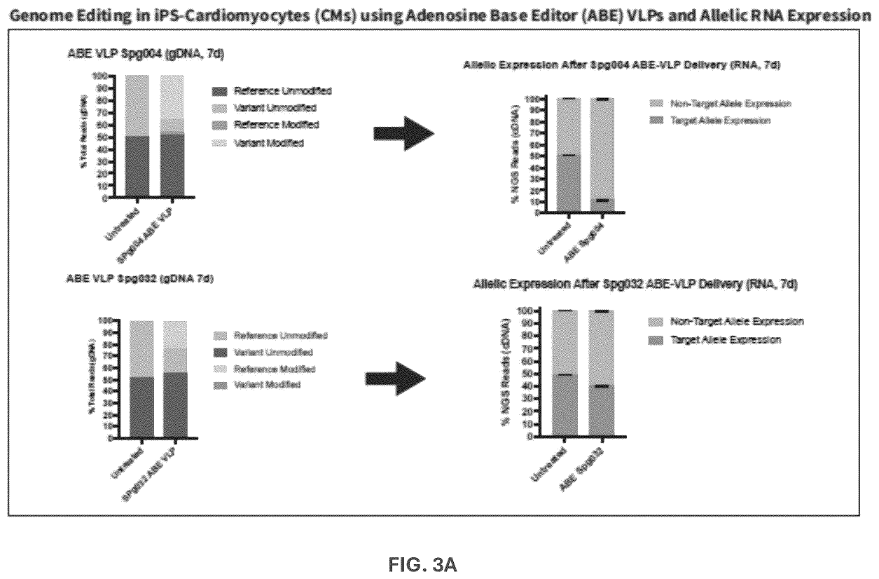

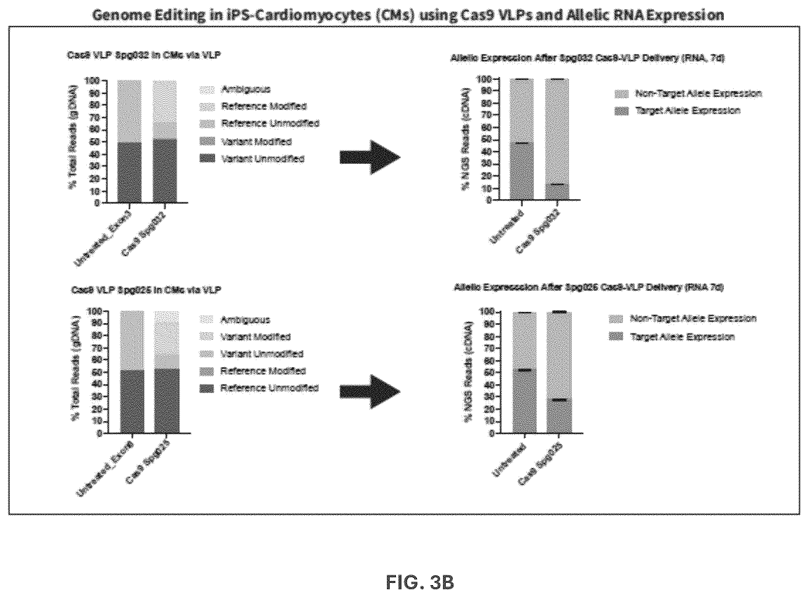

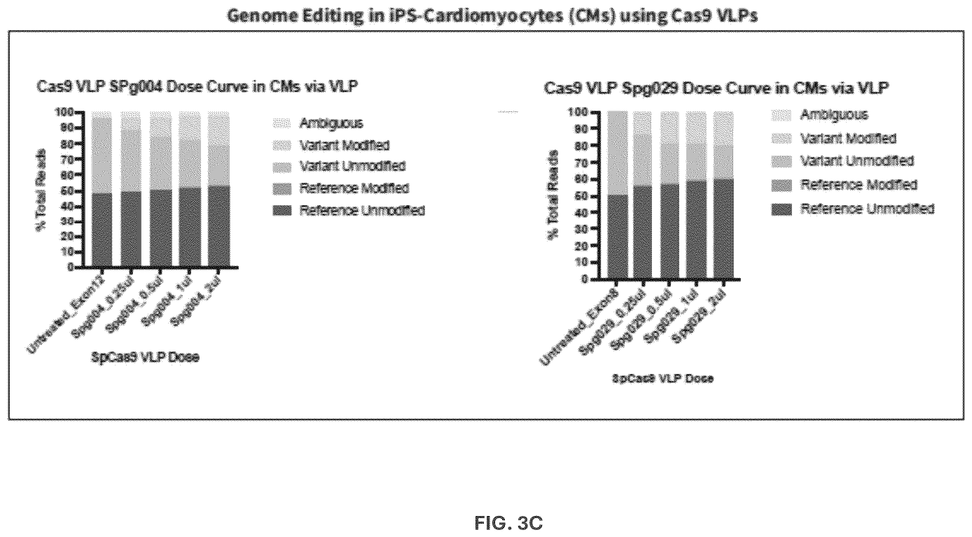

The lower right panel shows results for Spg018, a reference-targeting guide directed to the reference allele of rs735711. Untransfected cells show approximately equal proportions of non-target unmodified and target unmodified reads. Upon transfection with Spg018, modified reads appear at the target (reference) allele, while the non-target (alternate) allele remains largely unmodified. Together, the four panels of FIG. 1 demonstrate that guide RNAs Spg022, Spg023, Spg008, and Spg018 each edit the intended target allele with selectivity over the non-target allele in iPSC-derived cardiomyocytes.

FIG. 2 Demonstrates allele-specificity of the gRNAs (SPg004, SPg024, SPg025, SPg026, SPg029, SPg030, SPg030) in iPSCs. Allele-specificity of guide RNAs targeting SNPs rs2231126 and rs2069542 in iPSCs is shown. Data were generated through Illumina next-generation sequencing, and results are presented as percentage of total reads categorized as non-target unmodified (blue), target unmodified (red), and modified (gray).

The left panel shows results for SNP rs2231126. In untreated cells, reads are distributed between non-target unmodified and target unmodified categories, with no modified reads detected. Upon treatment with SPg004, a variant-targeting guide directed to rs2231126, modified reads appear, representing editing at the target allele, while the non-target allele remains largely unmodified.

The right panel shows results for SNP rs2069542. In untreated cells, reads are distributed between non-target unmodified and target unmodified categories, with no modified reads detected. Results are shown for multiple guide RNAs: SPg024 (reference-targeting), SPg025 (variant-targeting), SPg026 (reference-targeting), SPg029 (variant-targeting), SPg030 (reference-targeting), and SPg031 (variant-targeting). For each variant-targeting guide (SPg025, SPg029, SPg031), modified reads appear at the target (alternate) allele while the non-target (reference) allele remains largely unmodified. For each reference-targeting guide (SPg024, SPg026, SPg030), modified reads appear at the target (reference) allele while the non-target (alternate) allele remains largely unmodified. Together, the panels of FIG. 2 demonstrate that the tested guide RNAs exhibit allele-specific editing at both the rs2231126 and rs2069542 SNP sites in iPSCs.

FIGS. 3A-3C. A) Genome editing in iPSC-derived cardiomyocytes (CMs) using adenosine base editor (ABE) virus-like particles (VLPs) and allelic RNA expression analysis. Left panels show genomic DNA (gDNA) editing at day 7 following ABE-VLP delivery of Spg004 (upper) and Spg032 (lower), with reads categorized as reference unmodified, variant unmodified, reference modified, and variant modified. Right panels show allelic RNA expression after ABE-VLP delivery, with next-generation sequencing (NGS) reads from cDNA quantifying target allele expression and non-target allele expression for each guide. Arrows indicate the correspondence between gDNA editing and resulting allelic expression changes. B) Genome editing in iPSC-derived cardiomyocytes (CMs) using Cas9 virus-like particles (VLPs) and allelic RNA expression analysis. Left panels show genomic DNA editing following Cas9-VLP delivery of Spg032 (upper) and Spg025 (lower) in CMs, with reads categorized as ambiguous, variant modified, variant unmodified, reference modified, and reference unmodified. Right panels show allelic RNA expression after Cas9-VLP delivery, with NGS reads from cDNA quantifying target allele expression and non-target allele expression for each guide. Conditions shown include treated samples and pre-treatment controls. Arrows indicate the correspondence between gDNA editing and resulting allelic expression changes. C) Dose-response curves for genome editing in iPSC-derived cardiomyocytes (CMs) using Cas9 VLPs. The left panel shows a dose curve for Cas9-VLP delivery of Spg004 across multiple SpCas9 VLP doses, with total reads from gDNA categorized as ambiguous, variant modified, variant unmodified, reference modified, and reference unmodified, compared to an untreated control. The right panel shows a dose curve for Cas9-VLP delivery of Spg029 across multiple SpCas9 VLP doses, with reads categorized in the same manner. Data were generated through next-generation sequencing.

The data in the Figures demonstrates that that the use these gRNAs in combination with an enzyme, such as, either SpCas9 or an adenosine base editor (ABE8e), in a cardiomyocytes, highly efficient allele-specific editing can be achieved, as well as significant reduction in RNA expression of the target allele.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Most gene editing efforts have focused on correcting the rare disease-causing mutations, which can be effective for specific mutations, but are impractical when there are many different disease-causing mutations such as with MYH7. The approach provided herein differs from the mutation-specific approach in that the entire haplotype where the pathological mutation is targeted, rather than the mutation itself, with common heterozygous SNPs that are in cis with the disease mutation.

By targeting a commonly inherited SNP in the protein coding portion of the gene, resulting in Nonsense-Mediated Decay (NMD) of a mutant allele containing any pathogenic variant(s) can be instigated. Targeting a common SNP allows one to treat a higher proportion of the population, as a mutation agnostic approach allows one to maximize the proportion of targetable individuals one can treat. For instance, correcting a single disease mutation requires specific CRISPR reagents that would require FDA approval, yet could only be used in 0.5% of the MYH7 patients. In contrast, targeting a common SNP could target >20% of the MYH7 patients, which is 40-fold more patients for a single set of CRISPR reagents. This 40-fold increase in patients, makes the expense of FDA approval much more practical.

Provided herein are guides and the paired Cas ortholog that enable genomic indels (insertions or deletions) in MYH7, thereby introducing a premature stop-codon to disrupt expression of a diseased allele, consequently treating dominant-negative cardiomyopathy, while allowing only the expression of the wild-type allele. There are two broad classes of experimental approaches which would enable this strategy: mechanistic validation and delivery. First, the formation of an indel in cis with MYH7 mutations causing HCM is intended to instigate Nonsense-Mediated Decay, a process by which RNA transcripts with early stop codons are actively degraded by the cell.

The second broad class of enabling technologies is reagent delivery to the cell-type of interest. Currently, lipid nanoparticles (LNP) are used in various cell types and deliver mRNA molecules of the guide and Cas protein, to be expressed upon cell entry. Currently, LNPs need to be chemically synthesized and designed to target a tissue specifically. Thus, cardiomyocyte-specific LNPs would need to be screened in vivo for proper and specific delivery. Adeno-associated viruses, on the other hand, deliver ssDNA, have broad tissue specificity and are long lasting. The broad tissue-specificity and cellular persistence are areas to correct. To achieve tissue-specificity, tissue specific promoters can be used. To decrease persistence, there are methods for Cas9 self-inactivation.

Myosin-7 is a protein that in humans is encoded by the MYH7 (myosin, heavy polypeptide 7) gene. Mutations in MYH7 have been associated with inherited cardiomyopathies. Lowrance et al. were the first to identify the causative mutation Arg403Gln for hypertrophic cardiomyopathy (HCM) in the MYH7 gene (Geisterfer-Lowrance A A, et al. 1990. Cell. 62 (5): 999-1006). Other Studies have since identified more MYH7 mutations, that are estimated to be causal in approximately 40% of HCM cases. This condition is an autosomal-dominant disease, in which a single copy of the variant gene causes enlargement of the left ventricle of the heart. Disease onset usually occurs later in life, perhaps triggered by changes in thyroid hormone function and/or physical stress.

The human MYH7 gene is located on chromosome 14 (23.41-23.44 Mb) and has 40 exons.

A sequence for human MYH7 protein is available at NCBI accession no. NP_000248, shown below as SEQ ID NO: 7.

| | |
|---|---|
| 1 | MGDSEMAVFG AAAPYLRKSE KERLEAQTRP FDLKKDVFVP DDKQEFVKAK IVSREGGKVT |
| 61 | AETEYGKTVT VKEDQVMQQN PPKFDKIEDM AMLTFLHEPA VLYNLKDRYG SWMIYTYSGL |
| 121 | FCVTVNPYKW LPVYTPEVVA AYRGKKRSEA PPHIFSISDN AYQYMLTDRE NQSILITGES |
| 181 | GAGKTVNTKR VIQYFAVIAA IGDRSKKDQS PGKGTLEDQI IQANPALEAF GNAKTVRNDN |
| 241 | SSRFGKFIRI HFGATGKLAS ADIETYLLEK SRVIFQLKAE RDYHIFYQIL SNKKPELLDM |
| 301 | LLITNNPYDY AFISQGETTV ASIDDAEELM ATDNAFDVLG FTSEEKNSMY KLTGAIMHFG |
| 361 | NMKFKLKQRE EQAEPDGTEE ADKSAYLMGL NSADLLKGLC HPRVKVGNEY VTKGQNVQQV |
| 421 | IYATGALAKA VYERMFNWMV TRINATLETK QPRQYFIGVL DIAGFEIFDF NSFEQLCINF |
| 481 | TNEKLQQFFN HHMFVLEQEE YKKEGIEWTF IDFGMDLQAC IDLIEKPMGI MSILEEECMF |
| 541 | PKATDMTFKA KLFDNHLGKS ANFQKPRNIK GKPEAHFSLI HYAGIVDYNI IGWLQKNKDP |
| 601 | LNETVVGLYQ KSSLKLLSTL FANYAGADAP IEKGKGKAKK GSSFQTVSAL HRENLNKLMT |
| 661 | NLRSTHPHFV RCIIPNETKS PGVMDNPLVM HQLRCNGVLE GIRICRKGFP NRILYGDFRQ |
| 721 | RYRILNPAAI PEGQFIDSRK GAEKLLSSLD IDHNQYKFGH TKVFFKAGLL GLLEEMRDER |
| 781 | LSRIITRIQA QSRGVLARME YKKLLERRDS LLVIQWNIRA FMGVKNWPWM KLYFKIKPLL |
| 841 | KSAEREKEMA SMKEEFTRLK EALEKSEARR KELEEKMVSL LQEKNDLQLQ VQAEQDNLAD |
| 901 | AEERCDQLIK NKIQLEAKVK EMNERLEDEE EMNAELTAKK RKLEDECSEL KRDIDDLELT |
| 961 | LAKVEKEKHA TENKVKNLTE EMAGLDEIIA KLTKEKKALQ EAHQQALDDL QAEEDKVNTL |
| 1021 | TKAKVKLEQQ VDDLEGSLEQ EKKVRMDLER AKRKLEGDLK LTQESIMDLE NDKQQLDERL |
| 1081 | KKKDFELNAL NARIEDEQAL GSQLQKKLKE LQARIEELEE ELEAERTARA KVEKLRSDLS |
| 1141 | RELEEISERL EEAGGATSVQ IEMNKKREAE FQKMRRDLEE ATLQHEATAA ALRKKHADSV |
| 1201 | AELGEQIDNL QRVKQKLEKE KSEFKLELDD VTSNMEQIIK AKANLEKMCR TLEDQMNEHR |
| 1261 | SKAEETQRSV NDLTSQRAKL QTENGELSRQ LDEKEALISQ LTRGKLTYTQ QLEDLKRQLE |
| 1321 | EEVKAKNALA HALQSARHDC DLLREQYEEE TEAKAELQRV LSKANSEVAQ WRTKYETDAI |
| 1381 | QRTEELEEAK KKLAQRLQEA EEAVEAVNAK CSSLEKTKHR LQNEIEDLMV DVERSNAAAA |
| 1441 | ALDKKQRNFD KILAEWKQKY EESQSELESS QKEARSLSTE LFKLKNAYEE SLEHLETFKR |
| 1501 | ENKNLQEEIS DLTEQLGSSG KTIHELEKVR KQLEAEKMEL QSALEEAEAS LEHEEGKILR |
| 1561 | AQLEFNQIKA EIERKLAEKD EEMEQAKRNH LRVVDSLQTS LDAETRSRNE ALRVKKKMEG |
| 1621 | DLNEMEIQLS HANRMAAEAQ KQVKSLQSLL KDTQIQLDDA VRANDDLKEN IAIVERRNNL |
| 1681 | LQAELEELRA VVEQTERSRK LAEQELIETS ERVQLLHSQN TSLINQKKKM DADLSQLQTE |
| 1741 | VEEAVQECRN AEEKAKKAIT DAAMMAEELK KEQDTSAHLE RMKKNMEQTI KDLQHRLDEA |
| 1801 | EQIALKGGKK QLQKLEARVR ELENELEAEQ KRNAESVKGM RKSERRIKEL TYQTEEDRKN |
| 1861 | LLRLQDLVDK LQLKVKAYKR QAEEAEEQAN TNLSKFRKVQ HELDEAEERA DIAESQVNKL |
| 1921 | RAKSRDIGTK GLNEE |

A human cDNA encoding MYH7 protein with the SEQ ID NO: 7 sequence is available from the NCBI database as accession no. NM_000257 and provided below as SEQ ID NO: 8

| | |
|---|---|
| 1 | acagctctgt cctgctctgt gtctttccct gctgctctca ggtcccctgc aggccttggc |
| 61 | ccctttcctc atctgtagac acacttgagt agcccaggca cagccatggg agattcggag |
| 121 | atggcagtct ttggggctgc cgccccctac ctgcgcaagt cagagaagga gcggctagaa |
| 181 | gcgcagacca ggccttttga cctcaagaag gatgtcttcg tgcctgatga caaacaggag |
| 241 | tttgtcaagg ccaagatcgt gtctcgagag ggtggcaaag tcactgccga gaccgagtat |
| 301 | ggcaagacag tgaccgtgaa ggaggaccag gtgatgcagc agaacccacc caagttcgac |
| 361 | aaaatcgagg acatggccat gctgaccttc ctgcatgagc ccgcggtgct ctacaacctc |
| 421 | aaggatcgct acggctcctg gatgatctac acctactcgg gcctcttctg tgtcaccgtc |
| 481 | aacccttaca agtggctgcc ggtgtacact cctgaggtgg tggctgccta ccggggcaag |
| 541 | aagaggagcg aggccccgcc ccacatcttc tccatctccg acaacgccta tcagtacatg |
| 601 | ctgacagaca gagaaaacca gtccatcctg atcaccggag aatccggagc agggaagaca |
| 661 | gtcaacacca agagggtcat ccagtacttt gctgttattg cagccattgg ggaccgcagc |
| 721 | aagaaggacc agagcccggg caagggcacc ctggaggacc agatcatcca ggccaaccct |
| 781 | gctctggagg cctttggcaa tgccaagacc gtccggaacg acaactcctc ccgcttcggg |
| 841 | aaattcattc gaattcattt tggggcaaca ggaaagttgg catctgcaga catagagacc |
| 901 | tatcttctgg aaaaatccag agttattttc cagctgaaag cagagagaga ttatcacatt |
| 961 | ttctaccaaa tcctgtctaa caaaaagcct gagctgctgg acatgctgct gatcaccaac |
| 1021 | aacccctacg attatgcatt catctcccaa ggagagacca ccgtggcctc cattgatgac |
| 1081 | gctgaggagc tcatggccac tgataacgct tttgatgtgc tgggcttcac ttcagaggag |
| 1141 | aaaaactcca tgtataagct gacaggcgcc atcatgcact ttggaaacat gaagttcaag |
| 1201 | ctgaagcagc gggaggagca ggcggagcca gacggcactg aagaggctga caagtctgcc |
| 1261 | tacctcatgg ggctgaactc agccgacctg ctcaaggggc tgtgccaccc tcgggtgaaa |
| 1321 | gtgggcaatg agtacgtcac caaggggcag aatgtccagc aggtgatata tgccactggg |
| 1381 | gcactggcca aggcagtgta tgagaggatg ttcaactgga tggtgacgcg catcaatgcc |
| 1441 | accctggaga ccaagcagcc acgccagtac ttcataggag tcctggacat cgctggcttc |
| 1501 | gagatcttcg atttcaacag ctttgagcag ctctgcatca acttcaccaa cgagaagctg |
| 1561 | cagcagttct tcaaccacca catgtttgtg ctggagcagg aggagtacaa gaaggagggc |
| 1621 | atcgagtgga cattcattga ctttggcatg gacctgcagg cctgcattga cctcatcgag |
| 1681 | aagcccatgg gcatcatgtc catcctggaa gaggagtgca tgttccccaa ggccaccgac |
| 1741 | atgaccttca aggccaagct gtttgacaac cacctgggca aatccgccaa cttccagaag |
| 1801 | ccacgcaata tcaaggggaa gcctgaagcc cacttctccc tgatccacta tgccggcatc |
| 1861 | gtggactaca acatcattgg ctggctgcag aagaacaagg atcctctcaa tgagactgtc |
| 1921 | gtgggcttgt atcagaagtc ttccctcaag ctgctcagca ccctgtttgc caactatgct |
| 1981 | ggggctgatg cgcctattga gaagggcaaa ggcaaggcca agaaaggctc gtcctttcag |
| 2041 | actgtgtcag ctctgcacag ggaaaatctg aacaagctga tgaccaactt gcgctccacc |
| 2101 | catccccact ttgtacgttg tatcatccct aatgagacaa agtctccagg ggtgatggac |
| 2161 | aaccccctgg tcatgcacca gctgcgctgc aatggtgtgc tggagggcat ccgcatctgc |
| 2221 | aggaaaggct tccccaaccg catcctctac ggggacttcc ggcagaggta tcgcatcctg |
| 2281 | aacccagcgg ccatccctga gggacagttc attgatagca ggaagggggc agagaagctg |
| 2341 | ctcagctccc tggacattga tcacaaccag tacaagtttg gccacaccaa ggtgttcttc |
| 2401 | aaggccgggc tgctggggct gctggaggaa atgagggacg agaggctgag ccgcatcatc |
| 2461 | acgcgtatcc aggcccagtc ccgaggtgtg ctcgccagaa tggagtacaa aaagctgctg |
| 2521 | gaacgtagag actccctgct ggtaatccag tggaacattc gggccttcat gggggtcaag |
| 2581 | aattggccct ggatgaagct ctacttcaag atcaagccgc tgctgaagag tgcagaaaga |
| 2641 | gagaaggaga tggcctccat gaaggaggag ttcacacgcc tcaaagaggc gctagagaag |
| 2701 | tccgaggctc gccgcaagga gctggaggag aagatggtgt ccctgctgca ggagaagaat |
| 2761 | gacctgcagc tccaagtgca ggcggaacaa gacaacctgg cagatgctga ggagcgctgt |
| 2821 | gatcagctga tcaaaaacaa gattcagctg gaggccaagg tgaaggagat gaacgagagg |
| 2881 | ctggaggatg aggaggagat gaatgctgag ctcactgcca agaagcgcaa gctggaagat |
| 2941 | gagtgctcag agctcaaaag ggacatcgat gatctggagc tgacactggc caaagtggag |
| 3001 | aaggagaaac acgcaacaga gaacaaggtg aaaaacctga cagaggagat ggctgggctg |
| 3061 | gatgagatca ttgccaagct gaccaaggag aagaaagctc tgcaagaggc ccaccaacag |
| 3121 | gctctggatg accttcaggc cgaggaggac aaggtcaaca ccctgactaa ggccaaagtc |
| 3181 | aagctggagc agcaagtgga tgatctggaa ggatccctgg agcaagagaa gaaggtgcgc |
| 3241 | atggacctgg agcgagcgaa gcggaagctg gagggcgacc tgaagctgac ccaggagagc |
| 3301 | atcatggacc tggagaatga caagcagcag ctggatgagc ggctgaaaaa aaaagacttt |
| 3361 | gagctgaatg ctctcaacgc aaggattgag gatgaacagg ccctcggcag ccagctgcag |
| 3421 | aagaagctca aggagcttca ggcacgcatc gaggagctgg aggaggagct ggaggccgag |
| 3481 | cgcaccgcca gggctaaggt ggagaagctg cgctcagacc tgtctcggga gctggaggag |
| 3541 | atcagcgagc ggctggaaga ggccggcggg gccacgtccg tgcagatcga gatgaacaag |
| 3601 | aagcgcgagg ccgagttcca gaagatgcgg cgggacctgg aggaggccac gctgcagcac |
| 3661 | gaggccactg ccgcggccct gcgcaagaag cacgccgaca gcgtggccga gctgggcgag |
| 3721 | cagatcgaca acctgcagcg ggtgaagcag aagctggaga aggagaagag cgagttcaag |
| 3781 | ctggagctgg atgacgtcac ctccaacatg gagcagatca tcaaggccaa ggctaacctg |
| 3841 | gagaagatgt gccggacctt ggaagaccag atgaatgagc accggagcaa ggcggaggag |
| 3901 | acccagcgtt ctgtcaacga cctcaccagc cagcgggcca agttgcaaac cgagaatggt |
| 3961 | gagctgtccc ggcagctgga tgagaaggag gcactgatct cccagctgac ccgaggcaag |
| 4021 | ctcacctaca cccagcagct ggaggacctc aagaggcagc tggaggagga ggttaaggcg |
| 4081 | aagaacgccc tggcccacgc actgcagtcg gcccggcatg actgcgacct gctgcgggag |
| 4141 | cagtacgagg aggagacgga ggccaaggcc gagctgcagc gcgtcctttc caaggccaac |
| 4201 | tcggaggtgg cccagtggag gaccaagtat gagacggacg ccattcagcg gactgaggag |
| 4261 | ctcgaggagg ccaagaagaa gctggcccag cggctgcagg aagctgagga ggccgtggag |
| 4321 | gctgttaatg ccaagtgctc ctcgctggag aagaccaagc accggctaca gaatgagatc |
| 4381 | gaggacttga tggtggacgt agagcgctcc aatgctgctg ctgcagccct ggacaagaag |
| 4441 | cagaggaact tcgacaagat cctggccgag tggaagcaga agtatgagga gtcgcagtcg |
| 4501 | gagctggagt cctcgcagaa ggaggctcgc tccctcagca cagagctctt caaactcaag |
| 4561 | aacgcctatg aggagtccct ggaacatctg gagaccttca agcgggagaa caaaaacctg |
| 4621 | caggaggaga tctccgactt gactgagcag ttgggttcca gcggaaagac tatccatgag |
| 4681 | ctggagaagg tccgaaagca gctggaggcc gagaagatgg agctgcagtc agccctggag |
| 4741 | gaggccgagg cctccctgga gcacgaggag ggcaagatcc tccgggccca gctggagttc |
| 4801 | aaccagatca aggcagagat cgagcggaag ctggcagaga aggacgagga gatggaacag |
| 4861 | gccaagcgca accacctgcg ggtggtggac tcgctgcaga cctccctgga cgcagagaca |
| 4921 | cgcagccgca acgaggccct gagggtgaag aagaagatgg aaggagacct caatgagatg |
| 4981 | gagatccagc tcagccacgc caaccgcatg gccgccgagg cccagaagca agtcaagagc |
| 5041 | ctccagagct tgttgaagga cacccagatt cagctggacg atgcagtccg tgccaacgac |
| 5101 | gacctgaagg agaacatcgc catcgtggag cggcgcaaca acctgctgca ggctgagctg |
| 5161 | gaggagttgc gtgccgtggt ggagcagaca gagcggtccc ggaagctggc ggagcaggag |
| 5221 | ctgattgaga ctagtgagcg ggtgcagctg ctgcattccc agaacaccag cctcatcaac |
| 5281 | cagaagaaga agatggatgc tgacctgtcc cagctccaga ctgaagtgga ggaggcagtg |
| 5341 | caggagtgca ggaatgctga ggagaaggcc aagaaggcca tcacggatgc cgccatgatg |
| 5401 | gcagaggagc tgaagaagga gcaggacacc agcgcccacc tggagcgcat gaagaagaac |
| 5461 | atggaacaga ccattaagga cctgcagcac cggctggacg aagccgagca gatcgccctc |
| 5521 | aagggcggca agaagcagct gcagaagctg gaagcgcggg tgcgggagct ggagaatgag |
| 5581 | ctggaggccg agcagaagcg caacgcagag tcggtgaagg gcatgaggaa gagcgagcgg |
| 5641 | cgcatcaagg agctcaccta ccagacggag gaggacagga aaaacctgct gcggctgcag |
| 5701 | gacctggtag acaagctgca gctaaaggtc aaggcctaca agcgccaggc cgaggaggcg |
| 5761 | gaggagcaag ccaacaccaa cctgtccaag ttccgcaagg tgcagcacga gctggatgag |
| 5821 | gcagaggagc gggcggacat cgccgagtcc caggtcaaca agctgcgggc caagagccgt |
| 5881 | gacattggca cgaagggctt gaatgaggag tagctttgcc acatcttgat ctgctcagcc |
| 5941 | ctggaggtgc cagcaaagcc ccatgctgga gcctgtgtaa cagctccttg ggaggaagca |
| 6001 | gaataaagca attttccttg aagccga |

Sequence variations and mutations can naturally occur. For example, isoforms, variants, and mutants of the proteins and nucleic acids described herein can be detected and modified using the methods and compositions described herein. The isoforms, variants, and mutants can, for example, have sequences with between 55-100% sequence identity to a reference FUS sequence, for example with at least 55% sequence identity, at least 60%, at least 70%, at least 80%, at least 90%, at least 95%, at least 96%, at least 97%, at least 98%, or at least 99% sequence identity to any of the sequences described herein.

### Therapeutic Methods

A therapeutic method is described herein involving deleting the mutant allele while preserving the normal allele to promote development and maintenance of healthy cardiac tissue.

In the field of gene editing, it is commonly proposed to use a mutation as its own target for CRISPR-Cas9 based knockout of a mutant allele. However, this approach is untenable with MYH7-related diseases and conditions as over 200 different mutations have been identified in resulting in cardiomyopathy. Not only would the time and cost required to develop, optimize, and undergo FDA testing of individual guide RNAs (gRNAs) for each mutation be prohibitive, but some of these mutations are not directly targetable by a CRISPR-Cas9 gRNAs in the first place. Second, when using any editing strategy that targets these mutations and introduces an insertion or deletion (indel), the gene risks creating a new, and potentially worse disease-causing mutation.

As mentioned, over 200 different MYH7 mutations cause cardiomyopathies. Since only one allele of MYH7 is required for normal function, eliminating disease alleles is curative. A key challenge of current technologies is the diversity of MYH7 disease mutations, making it difficult (or impossible) to target each disease allele.

However, the compositions and methods described herein target a commonly inherited SNP early in the protein coding portion of the gene, leading to the instigation of Nonsense-Mediated Decay (NMD) of the mutant allele containing any pathogenic single nucleotide variant. In particular, the guides and paired Cas ortholog enable genomic indels (insertions or deletions) in MYH7, thereby introducing a premature stop-codon to disrupt expression of a diseased allele, consequently treating dominant-negative cardiomyopathy.

Provided herein are SpCas9 guide RNAs (gRNAs) targeting common SNPs that are in protein-coding exons of MYH7, a gene that harbors >200 disease-causing mutations that result in a lethal cardiomyopathy. Targeting a commonly inherited, allele-specific SNP in a diseased cell disrupts the expression of a mutant allele, while preserving the expression of the wild-type allele, thereby reversing disease phenotypes or preventing disease progression. In Table 1, gRNA sequences are described (are listed 5′->3′). Other Cas species can be used to target these same SNPs.

Sequences gRNAs are shown in Table 1 that target SNP alleles (SEQ ID NOS: 1-6 and 13-14).

TABLE 1
Allele-Specific SpCas9 gRNAs

| Guide Name | SNP Targeted | Sequence (5′→′) |
|---|---|---|
| Spg022 | rs2069540 (alternate allele) | CACCTTGCCATACTCAGTCT |
| | | (SEQ ID NO: 1) |
| Spg023 | rs2069540(reference allele) | CACCTTGCCATACTCGGTCT |
| | | (SEQ ID NO: 2) |
| Spg008 | rs735711 (alternate allele) | GAAGTTCAAACTGAAGCAGC |
| | | (SEQ ID NO: 3) |
| Spg018 | rs735711 (reference allele) | GAAGTTCAAGCTGAAGCAGC |
| | | (SEQ ID NO: 4) |
| Spg010 | rs735711 (alternate allele) | TGAAGTTCAAACTGAAGCAG |
| | | (SEQ ID NO: 5) |
| Spg019 | rs735711 (reference allele) | TGAAGTTCAAGCTGAAGCAG |
| | | (SEQ ID NO: 6) |

| Short | | | | |
|---|---|---|---|---|
| Guide | SNP | Allele | | |
| Name | Targeted | Targeted | Full Guide Name | Sequence (5'-3') |
| Spg003 | rs2231126 | Reference | MYH7_rs2231126_Reference_g1 | CTCCCACCTTCAGTGCCGTC |
| | | | | (SEQ ID NO: 13) |
| Spg004 | rs2231126 | Alternate | MYH7_rs2231126_Alternate_g1 | CTCCCACCTTCAGTGCCaTC |
| | | | | (SEQ ID NO: 14) |
| Spg005 | rs2231126 | Reference | MYH7_rs2231126_Reference_g2 | GCGGAGCCAGACGGCACTGA |
| | | | | (SEQ ID NO: 15) |
| Spg006 | rs2231126 | Alternate | MYH7_rs2231126_Alternate_g2 | GCGGAGCCAGAtGGCACTG |
| | | | | (SEQ ID NO: 16) |
| Spg024 | rs2069542 | Reference | MYH7_rs2069542_Reference_g1 | GTCAGGGACCACTCACGAAG |
| | | | | (SEQ ID NO: 17) |
| Spg025 | rs2069542 | Alternate | MYH7_rs2069542_Alternate_g1 | GTCAGGGACCACTCACaAAG |
| | | | | (SEQ ID NO: 18) |
| Spg026 | rs2069542 | Reference | MYH7_rs2069542_Reference_g2 | TCAGGGACCACTCACGAAG |
| | | | | (SEQ ID NO: 19) |
| Spg027 | rs2069542 | Alternate | MYH7_rs2069542_Alternate_g2 | TCAGGGACCACTCACaAAGC |
| | | | | (SEQ ID NO: 20) |
| Spg028 | rs2069542 | Reference | MYH7_rs2069542_Reference_g3 | GGGACCACTCACGAAGCGGG |
| | | | | (SEQ ID NO: 21) |
| Spg029 | rs2069542 | Alternate | MYH7_rs2069542_Alternate_g3 | GGGACCACTCACaAAGCGGG |
| | | | | (SEQ ID NO: 22) |
| Spg030 | rs2069542 | Reference | MYH7_rs2069542_Reference_g4 | AACTCCTCCCGCTTCGTGAG |
| | | | | (SEQ ID NO: 23) |
| Spg031 | rs2069542 | Alternate | MYH7_rs2069542_Alternate_g4 | AACTCCTCCCGCTTtGTGAG |
| | | | | (SEQ ID NO: 24) |
| Spg032 | rs2069540 | | MYH7_rs2069540_Reference_g2 | GACACCCACCTTGCCATACT |
| | | | | (SEQ ID NO: 25) |

FIG. 1 demonstrates the efficiency of editing a target MYH7 allele using 5 allele-specific guides in iPSC-derived cardiomyocytes (Spg022, Spg023, Spg008, Spg010, Spg018, and Spg019). Three common SNPs can be targeted (rs2069540, rs735711, rs7157716). Data provided herein are from rs2069540 and rs735711. These data were generated through Next-generation sequencing.

FIG. 2 discloses data for SNPs rs2231126 and rs2069542. These data were generated in iPSCs but demonstrate the allele-specificity of the guide RNAs. These data were generated through Next-generation sequencing.

### Gene Editing Technology

Described herein are allele-specific guide RNAs that can repair, knockdown or knockout the expression of an undesired polypeptide encoded by a variant (mutant) MYH7 allele. The CRISPR-Cas9 genome-editing system can be used to delete/correct MYH7 mutations that are correlated with cardiomyopathy. For example, one or two guide RNAs (gRNAs) can be used to recognize one or more target sequence in a subject's genome, and a corrective nuclease can act as a pair of scissors to cleave a single-strand or a double-strand of genomic DNA. Mutations in the genome that are near the cleavage site(s) can be repaired by an endogenous Non-Homologous End Joining (NHEJ) or Homology Directed Repair (HDR) repair pathway. Hence, the guide RNAs guide the corrective nuclease to cleave the targeted genomic site for deletion and/or repair by endogenous mechanisms. Examples of the specific guide RNA sequences provided herein are shown in Table 1.

The Cas system can recognize any sequence in the genome that matches 20 bases of a gRNA. However, each gRNA must also be adjacent to a “Protospacer Adjacent Motif” (PAM) in the genome, which is invariant for each type of Cas protein. The PAM sequence allows for the Cas protein to recognize the adjacent protospacer and to activate its nuclease activity. See Doudna et al., Science 346 (6213): 1077, 1258096 (2014); and Jinek et al., Science 337:816-21 (2012). Hence, the guide RNAs have a PAM site sequence that allows a Cas protein to bind to its target genomic location.

When the Cas system was first described for Cas9, with a “NGG” PAM site, the PAM was somewhat limiting in that it required a GG in the right orientation to the site to be targeted. Different Cas9 species have now been described with different PAM sites. See Jinek et al., Science 337:816-21 (2012); Ran et al., Nature 520:186-91 (2015); and Zetsche et al., Cell 163:759-71 (2015). In addition, mutations in the PAM recognition domain (Table 2) have increased the diversity of PAM sites for SpCas9 and SaCas9. See Kleinstiver et al., Nat Biotechnol 33:1293-1298 (2015); and Kleinstiver et al., Nature 523:481-5 (2015).

Table 2 summarizes information about PAM sites.

TABLE 2
PAM sites

| | PAM sites |
|---|---|
| SpCas9 | NGG |
| SpCas9 VRER variant | NGCG |
| SpCas9 EQR variant | NGAG |
| SpCas9 VQR variant | NGAN or NGNG |
| SaCas9 | NNGRRT |
| SaCas9, KKH variant | NNNRRT |
| FnCas2 (Cpf1) | TTN |

DNA annotations:
N = A, C, T or G
R = Purine, A or G

Note that the guide RNAs for SpCas9 and SaCas9 cover 20 bases in the 5′direction of the PAM site, while for FnCas12 (Cpf1) the guide RNA covers 20 bases to 3′ of the PAM.

There are a number of different types of corrective nucleases and systems that can be used for gene editing. The corrective nuclease employed can in some cases be any DNA binding protein with corrective nuclease activity. Examples of corrective nuclease include Streptococcus pyogenes Cas (SpCas9) nucleases, Staphylococcus aureus Cas9 (SaCas9) nucleases, Francisella novicida Cas12a (FnCas12a, also called dFnCpf1) nucleases, Zinc Finger Nucleases (ZFN), Meganuclease, Transcription activator-like effector nucleases (TALEN), Fok-I nucleases, any DNA binding protein with nuclease activity, any DNA binding protein bound to a corrective nuclease, or any combinations thereof. However, the CRISPR-Cas systems are generally the most widely used. In some cases, the corrective nuclease is therefore a Cas nuclease.

CRISPR-Cas systems are generally divided into two classes. The class 1 system contains types I, III and IV, and the class 2 system contains types II, V, and VI. The class 1 CRISPR-Cas system uses a complex of several Cas proteins, whereas the class 2 system only uses a single Cas protein with multiple domains. The class 2 CRISPR-Cas system is usually preferable for gene-engineering applications because of its simplicity and ease of use.

A variety of Cas nucleases can be employed in the methods described herein. Three species that have been best characterized are provided as examples. The most commonly used Cas nuclease is a Streptococcus pyogenes Cas9, (SpCas9). More recently described forms of Cas include Staphylococcus aureus Cas9 (SaCas9) and Francisella novicida Cas12a (FnCas12a, also called FnCpf1). Jinek et al., Science 337:816-21 (2012); Qi et al., Cell 152:1173-83 (2013); Ran et al., Nature 520:186-91 (2015); Zetsche et al., Cell 163:759-71 (2015).

One example of an amino acid sequence for Streptococcus pyogenes Cas9 (SpCas9) nuclease is provided below (SEQ ID NO: 9).

| | |
|---|---|
| 1 | MDKKYSIGLD IGTNSVGWAV ITDEYKVPSK KFKVLGNTDR |
| 41 | HSIKKNLIGA LLFDSGETAE ATRLKRTARR RYTRRKNRIC |
| 81 | YLQEIFSNEM AKVDDSFFHR LEESFLVEED KKHERHPIFG |
| 121 | NIVDEVAYHE KYPTIYHLRK KLVDSTDKAD LRLIYLALAH |
| 161 | MIKFRGHFLI EGDLNPDNSD VDKLFIQLVQ TYNQLFEENP |
| 201 | INASGVDAKA ILSARLSKSR RLENLIAQLP GEKKNGLFGN |
| 241 | LIALSLGLTP NFKSNFDLAE DAKLQLSKDT YDDDLDNLLA |
| 281 | QIGDQYADLF LAAKNLSDAI LLSDILRVNT EITKAPLSAS |
| 321 | MIKRYDEHHQ DLTLLKALVR QQLPEKYKEI FFDQSKNGYA |
| 361 | GYIDGGASQE EFYKFIKPIL EKMDGTEELL VKLNREDLLR |
| 401 | KQRTFDNGSI PHQIHLGELH AILRRQEDFY PFLKDNREKI |
| 441 | EKILTFRIPY YVGPLARGNS RFAWMTRKSE ETITPWNFEE |
| 481 | VVDKGASAQS FIERMINFDK NLPNEKVLPK HSLLYEYFTV |
| 521 | YNELTKVKYV TEGMRKPAFL SGEQKKAIVD LLFKTNRKVT |
| 561 | VKQLKEDYFK KIECFDSVEI SGVEDRFNAS LGTYHDLLKI |
| 601 | IKDKDFLDNE ENEDILEDIV LTLTLFEDRE MIEERLKTYA |
| 641 | HLFDDKVMKQ LKRRRYTGWG RLSRKLINGI RDKQSGKTIL |
| 681 | DFLKSDGFAN RNFMQLIHDD SLTFKEDIQK AQVSGQGDSL |
| 721 | HEHIANLAGS PAIKKGILQT VKVVDELVKV MGRHKPENIV |
| 761 | IEMARENQTT QKGQKNSRER MKRIEEGIKE LGSQILKEHP |
| 801 | VENTQLQNEK LYLYYLQNGR DMYVDQELDI NRLSDYDVDH |
| 841 | IVPQSFLKDD SIDNKVLTRS DKNRGKSDNV PSEEVVKKMK |
| 881 | NYWRQLLNAK LITQRKFDNL TKAERGGLSE LDKAGFIKRQ |
| 921 | LVETRQITKH VAQILDSRMN TKYDENDKLI REVKVITLKS |
| 961 | KLVSDFRKDF QFYKVREINN YHHAHDAYLN AVVGTALIKK |
| 1001 | YPKLESEFVY GDYKVYDVRK MIAKSEQEIG KATAKYFFYS |
| 1041 | NIMNFFKTEI TLANGEIRKR PLIETNGETG EIVWDKGRDF |
| 1081 | ATVRKVLSMP QVNIVKKTEV QTGGFSKESI LPKRNSDKLI |
| 1121 | ARKKDWDPKK YGGFDSPTVA YSVLVVAKVE KGKSKKLKSV |
| 1161 | KELLGITIME RSSFEKNPID FLEAKGYKEV KKDLIIKLPK |
| 1201 | YSLFELENGR KRMLASAGEL QKGNELALPS KYVNFLYLAS |
| 1241 | HYEKLKGSPE DNEQKQLFVE QHKHYLDEII EQISEFSKRV |
| 1281 | ILADANLDKV LSAYNKHRDK PIREQAENII HLFTLINLGA |
| 1321 | PAAFKYFDTT IDRKRYTSTK EVLDATLIHQ SITGLYETRI |
| 1361 | DLSQLGGD |

A cDNA that encodes the Streptococcus pyogenes Cas9 (SpCas9) is provided below (SEQ ID NO: 10).

| | |
|---|---|
| 1 | GACAAGAAGT ACAGCATCGG CCTGGACATC GGCACCAACT |
| 41 | CTGTGGGCTG GGCCGTGATC ACCGACGAGT ACAAGGTGCC |
| 81 | CAGCAAGAAA TTCAAGGTGC TGGGCAACAC CGACCGGCAC |
| 121 | AGCATCAAGA AGAACCTGAT CGGAGCCCTG CTGTTCGACA |
| 161 | GCGGCGAAAC AGCCGAGGCC ACCCGGCTGA AGAGAACCGC |
| 201 | CAGAAGAAGA TACACCAGAC GGAAGAACCG GATCTGCTAT |
| 241 | CTGCAAGAGA TCTTCAGCAA CGAGATGGCC AAGGTGGACG |
| 281 | ACAGCTTCTT CCACAGACTG GAAGAGTCCT TCCTGGTGGA |
| 321 | AGAGGATAAG AAGCACGAGC GGCACCCCAT CTTCGGCAAC |
| 361 | ATCGTGGACG AGGTGGCCTA CCACGAGAAG TACCCCACCA |
| 401 | TCTACCACCT GAGAAAGAAA CTGGTGGACA GCACCGACAA |
| 441 | GGCCGACCTG CGGCTGATCT ATCTGGCCCT GGCCCACATG |
| 481 | ATCAAGTTCC GGGGCCACTT CCTGATCGAG GGCGACCTGA |
| 521 | ACCCCGACAA CAGCGACGTG GACAAGCTGT TCATCCAGCT |
| 561 | GGTGCAGACC TACAACCAGC TGTTCGAGGA AAACCCCATC |
| 601 | AACGCCAGCG GCGTGGACGC CAAGGCCATC CTGTCTGCCA |
| 641 | GACTGAGCAA GAGCAGACGG CTGGAAAATC TGATCGCCCA |
| 681 | GCTGCCCGGC GAGAAGAAGA ATGGCCTGTT CGGAAACCTG |
| 721 | ATTGCCCTGA GCCTGGGCCT GACCCCCAAC TTCAAGAGCA |
| 761 | ACTTCGACCT GGCCGAGGAT GCCAAACTGC AGCTGAGCAA |
| 801 | GGACACCTAC GACGACGACC TGGACAACCT GCTGGCCCAG |
| 841 | ATCGGCGACC AGTACGCCGA CCTGTTTCTG GCCGCCAAGA |
| 881 | ACCTGTCCGA CGCCATCCTG CTGAGCGACA TCCTGAGAGT |
| 921 | GAACACCGAG ATCACCAAGG CCCCCCTGAG CGCCTCTATG |
| 961 | ATCAAGAGAT ACGACGAGCA CCACCAGGAC CTGACCCTGC |
| 1001 | TGAAAGCTCT CGTGCGGCAG CAGCTGCCTG AGAAGTACAA |
| 1041 | AGAGATTTTC TTCGACCAGA GCAAGAACGG CTACGCCGGC |
| 1081 | TACATTGACG GCGGAGCCAG CCAGGAAGAG TTCTACAAGT |
| 1121 | TCATCAAGCC CATCCTGGAA AAGATGGACG GCACCGAGGA |
| 1161 | ACTGCTCGTG AAGCTGAACA GAGAGGACCT GCTGCGGAAG |
| 1201 | CAGCGGACCT TCGACAACGG CAGCATCCCC CACCAGATCC |
| 1241 | ACCTGGGAGA GCTGCACGCC ATTCTGCGGC GGCAGGAAGA |
| 1281 | TTTTTACCCA TTCCTGAAGG ACAACCGGGA AAAGATCGAG |
| 1321 | AAGATCCTGA CCTTCCGCAT CCCCTACTAC GTGGGCCCTC |
| 1361 | TGGCCAGGGG AAACAGCAGA TTCGCCTGGA TGACCAGAAA |
| 1401 | GAGCGAGGAA ACCATCACCC CCTGGAACTT CGAGGAAGTG |
| 1441 | GTGGACAAGG GCGCTTCCGC CCAGAGCTTC ATCGAGCGGA |
| 1481 | TGACCAACTT CGATAAGAAC CTGCCCAACG AGAAGGTGCT |
| 1521 | GCCCAAGCAC AGCCTGCTGT ACGAGTACTT CACCGTGTAT |
| 1561 | AACGAGCTGA CCAAAGTGAA ATACGTGACC GAGGGAATGA |
| 1601 | GAAAGCCCGC CTTCCTGAGC GGCGAGCAGA AAAAGGCCAT |
| 1641 | CGTGGACCTG CTGTTCAAGA CCAACCGGAA AGTGACCGTG |
| 1681 | AAGCAGCTGA AAGAGGACTA CTTCAAGAAA ATCGAGTGCT |
| 1721 | TCGACTCCGT GGAAATCTCC GGCGTGGAAG ATCGGTTCAA |
| 1761 | CGCCTCCCTG GGCACATACC ACGATCTGCT GAAAATTATC |
| 1801 | AAGGACAAGG ACTTCCTGGA CAATGAGGAA AACGAGGACA |
| 1841 | TTCTGGAAGA TATCGTGCTG ACCCTGACAC TGTTTGAGGA |
| 1881 | CAGAGAGATG ATCGAGGAAC GGCTGAAAAC CTATGCCCAC |
| 1921 | CTGTTCGACG ACAAAGTGAT GAAGCAGCTG AAGCGGCGGA |
| 1961 | GATACACCGG CTGGGGCAGG CTGAGCCGGA AGCTGATCAA |
| 2001 | CGGCATCCGG GACAAGCAGT CCGGCAAGAC AATCCTGGAT |
| 2041 | TTCCTGAAGT CCGACGGCTT CGCCAACAGA AACTTCATGC |
| 2081 | AGCTGATCCA CGACGACAGC CTGACCTTTA AAGAGGACAT |
| 2121 | CCAGAAAGCC CAGGTGTCCG GCCAGGGCGA TAGCCTGCAC |
| 2161 | GAGCACATTG CCAATCTGGC CGGCAGCCCC GCCATTAAGA |
| 2201 | AGGGCATCCT GCAGACAGTG AAGGTGGTGG ACGAGCTCGT |
| 2241 | GAAAGTGATG GGCCGGCACA AGCCCGAGAA CATCGTGATC |
| 2281 | GAAATGGCCA GAGAGAACCA GACCACCCAG AAGGGACAGA |
| 2321 | AGAACAGCCG CGAGAGAATG AAGCGGATCG AAGAGGGCAT |
| 2361 | CAAAGAGCTG GGCAGCCAGA TCCTGAAAGA ACACCCCGTG |
| 2401 | GAAAACACCC AGCTGCAGAA CGAGAAGCTG TACCTGTACT |
| 2441 | ACCTGCAGAA TGGGCGGGAT ATGTACGTGG ACCAGGAACT |
| 2481 | GGACATCAAC CGGCTGTCCG ACTACGATGT GGACCATATC |
| 2521 | GTGCCTCAGA GCTTTCTGAA GGACGACTCC ATCGACAACA |
| 2561 | AGGTGCTGAC CAGAAGCGAC AAGAACCGGG GCAAGAGCGA |
| 2601 | CAACGTGCCC TCCGAAGAGG TCGTGAAGAA GATGAAGAAC |
| 2641 | TACTGGCGGC AGCTGCTGAA CGCCAAGCTG ATTACCCAGA |
| 2681 | GAAAGTTCGA CAATCTGACC AAGGCCGAGA GAGGCGGCCT |
| 2721 | GAGCGAACTG GATAAGGCCG GCTTCATCAA GAGACAGCTG |
| 2761 | GTGGAAACCC GGCAGATCAC AAAGCACGTG GCACAGATCC |
| 2801 | TGGACTCCCG GATGAACACT AAGTACGACG AGAATGACAA |
| 2841 | GCTGATCCGG GAAGTGAAAG TGATCACCCT GAAGTCCAAG |
| 2881 | CTGGTGTCCG ATTTCCGGAA GGATTTCCAG TTTTACAAAG |
| 2921 | TGCGCGAGAT CAACAACTAC CACCACGCCC ACGACGCCTA |
| 2961 | CCTGAACGCC GTCGTGGGAA CCGCCCTGAT CAAAAAGTAC |
| 3001 | CCTAAGCTGG AAAGCGAGTT CGTGTACGGC GACTACAAGG |
| 3041 | TGTACGACGT GCGGAAGATG ATCGCCAAGA GCGAGCAGGA |
| 3081 | AATCGGCAAG GCTACCGCCA AGTACTTCTT CTACAGCAAC |
| 3121 | ATCATGAACT TTTTCAAGAC CGAGATTACC CTGGCCAACG |
| 3161 | GCGAGATCCG GAAGCGGCCT CTGATCGAGA CAAACGGCGA |
| 3201 | AACCGGGGAG ATCGTGTGGG ATAAGGGCCG GGATTTTGCC |
| 3241 | ACCGTGCGGA AAGTGCTGAG CATGCCCCAA GTGAATATCG |
| 3281 | TGAAAAAGAC CGAGGTGCAG ACAGGCGGCT TCAGCAAAGA |
| 3321 | GTCTATCCTG CCCAAGAGGA ACAGCGATAA GCTGATCGCC |
| 3361 | AGAAAGAAGG ACTGGGACCC TAAGAAGTAC GGCGGCTTCG |
| 3401 | ACAGCCCCAC CGTGGCCTAT TCTGTGCTGG TGGTGGCCAA |
| 3441 | AGTGGAAAAG GGCAAGTCCA AGAAACTGAA GAGTGTGAAA |
| 3481 | GAGCTGCTGG GGATCACCAT CATGGAAAGA AGCAGCTTCG |
| 3521 | AGAAGAATCC CATCGACTTT CTGGAAGCCA AGGGCTACAA |
| 3561 | AGAAGTGAAA AAGGACCTGA TCATCAAGCT GCCTAAGTAC |
| 3601 | TCCCTGTTCG AGCTGGAAAA CGGCCGGAAG AGAATGCTGG |
| 3641 | CCTCTGCCGG CGAACTGCAG AAGGGAAACG AACTGGCCCT |
| 3681 | GCCCTCCAAA TATGTGAACT TCCTGTACCT GGCCAGCCAC |
| 3721 | TATGAGAAGC TGAAGGGCTC CCCCGAGGAT AATGAGCAGA |
| 3761 | AACAGCTGTT TGTGGAACAG CACAAGCACT ACCTGGACGA |
| 3801 | GATCATCGAG CAGATCAGCG AGTTCTCCAA GAGAGTGATC |
| 3841 | CTGGCCGACG CTAATCTGGA CAAAGTGCTG TCCGCCTACA |
| 3881 | ACAAGCACCG GGATAAGCCC ATCAGAGAGC AGGCCGAGAA |
| 3921 | TATCATCCAC CTGTTTACCC TGACCAATCT GGGAGCCCCT |
| 3961 | GCCGCCTTCA AGTACTTTGA CACCACCATC GACCGGAAGA |
| 4001 | GGTACACCAG CACCAAAGAG GTGCTGGACG CCACCCTGAT |
| 4041 | CCACCAGAGC ATCACCGGCC TGTACGAGAC ACGGATCGAC |
| 4081 | CTGTCTCAGC TGGGAGGCGA C |

An amino acid sequence for a Francisella novicida Cas12a (FnCas12a, also called FnCpf1) is shown below (SEQ ID NO: 11).

| | |
|---|---|
| 1 | MTQFEGFTNL YQVSKTLRFE LIPQGKTLKH IQEQGFIEED |
| 41 | KARNDHYKEL KPIIDRIYKT YADQCLQLVQ LDWENLSAAI |
| 81 | DSYRKEKTEE TRNALIEEQA TYRNAIHDYF IGRTDNLTDA |
| 121 | INKRHAEIYK GLFKAELFNG KVLKQLGTVT TTEHENALLR |
| 161 | SFDKFTTYFS GFYENRKNVF SAEDISTAIP HRIVQDNFPK |
| 201 | FKENCHIFTR LITAVPSLRE HFENVKKAIG IFVSTSIEEV |
| 241 | FSFPFYNQLL TQTQIDLYNQ LLGGISREAG TEKIKGLNEV |
| 281 | LNLAIQKNDE TAHIIASLPH RFIPLFKQIL SDRNTLSFIL |
| 321 | EEFKSDEEVI QSFCKYKTLL RNENVLETAE ALFNELNSID |
| 361 | LTHIFISHKK LETISSALCD HWDTLRNALY ERRISELTGK |
| 401 | ITKSAKEKVQ RSLKHEDINL QEIISAAGKE LSEAFKQKTS |
| 441 | EILSHAHAAL DQPLPTTLKK QEEKEILKSQ LDSLLGLYHL |
| 481 | LDWFAVDESN EVDPEFSARL TGIKLEMEPS LSFYNKARNY |
| 521 | ATKKPYSVEK FKLNFQMPTL ASGWDVNKEK NNGAILFVKN |
| 561 | GLYYLGIMPK QKGRYKALSF EPTEKTSEGF DKMYYDYFPD |
| 601 | AAKMIPKCST QLKAVTAHFQ THTTPILLSN NFIEPLEITK |
| 641 | EIYDLNNPEK EPKKFQTAYA KKTGDQKGYR EALCKWIDFT |
| 681 | RDFLSKYTKT TSIDLSSLRP SSQYKDLGEY YAELNPLLYH |
| 721 | ISFQRIAEKE IMDAVETGKL YLFQIYNKDF AKGHHGKPNL |
| 761 | HTLYWTGLFS PENLAKTSIK LNGQAELFYR PKSRMKRMAH |
| 801 | RLGEKMLNKK LKDQKTPIPD TLYQELYDYV NHRLSHDLSD |
| 841 | EARALLPNVI TKEVSHEIIK DRRFTSDKFF FHVPITLNYQ |
| 881 | AANSPSKFNQ RVNAYLKEHP ETPIIGIDRG ERNLIYITVI |
| 921 | DSTGKILEQR SLNTIQQFDY QKKLDNREKE RVAARQAWSV |
| 961 | VGTIKDLKQG YLSQVIHEIV DLMIHYQAVV VLENLNFGFK |
| 1001 | SKRTGIAEKA VYQQFEKMLI DKLNCLVLKD YPAEKVGGVL |
| 1041 | NPYQLTDQFT SFAKMGTQSG FLFYVPAPYT SKIDPLTGFV |
| 1081 | DPFVWKTIKN HESRKHFLEG FDFLHYDVKT GDFILHFKMN |
| 1121 | RNLSFQRGLP GFMPAWDIVF EKNETQFDAK GTPFIAGKRI |
| 1161 | VPVIENHRFT GRYRDLYPAN ELIALLEEKG IVFRDGSNIL |
| 1201 | PKLLENDDSH AIDTMVALIR SVLQMRNSNA ATGEDYINSP |
| 1241 | VRDLNGVCFD SRFQNPEWPM DADANGAYHI ALKGQLLLNH |
| 1281 | LKESKDLKLQ NGISNQDWLA YIQELRN |

A cDNA that encodes the foregoing Francisella novicida Cas12a (FnCas12a, also called dFnCpf1) polypeptide is shown below (SEQ ID NO: 12).

| | |
|---|---|
| 1 | ATGACACAGT TCGAGGGCTT TACCAACCTG TATCAGGTGA |
| 41 | GCAAGACACT GCGGTTTGAG CTGATCCCAC AGGGCAAGAC |
| 81 | CCTGAAGCAC ATCCAGGAGC AGGGCTTCAT CGAGGAGGAC |
| 121 | AAGGCCCGCA ATGATCACTA CAAGGAGCTG AAGCCCATCA |
| 161 | TCGATCGGAT CTACAAGACC TATGCCGACC AGTGCCTGCA |
| 201 | GCTGGTGCAG CTGGATTGGG AGAACCTGAG CGCCGCCATC |
| 241 | GACTCCTATA GAAAGGAGAA AACCGAGGAG ACAAGGAACG |
| 281 | CCCTGATCGA GGAGCAGGCC ACATATCGCA ATGCCATCCA |
| 321 | CGACTACTTC ATCGGCCGGA CAGACAACCT GACCGATGCC |
| 361 | ATCAATAAGA GACACGCCGA GATCTACAAG GGCCTGTTCA |
| 401 | AGGCCGAGCT GTTTAATGGC AAGGTGCTGA AGCAGCTGGG |
| 441 | CACCGTGACC ACAACCGAGC ACGAGAACGC CCTGCTGCGG |
| 481 | AGCTTCGACA AGTTTACAAC CTACTTCTCC GGCTTTTATG |
| 521 | AGAACAGGAA GAACGTGTTC AGCGCCGAGG ATATCAGCAC |
| 561 | AGCCATCCCA CACCGCATCG TGCAGGACAA CTTCCCCAAG |
| 601 | TTTAAGGAGA ATTGTCACAT CTTCACACGC CTGATCACCG |
| 721 | CCGTGCCCAG CCTGCGGGAG CACTTTGAGA ACGTGAAGAA |
| 761 | GGCCATCGGC ATCTTCGTGA GCACCTCCAT CGAGGAGGTG |
| 801 | TTTTCCTTCC CTTTTTATAA CCAGCTGCTG ACACAGACCC |
| 841 | AGATCGACCT GTATAACCAG CTGCTGGGAG GAATCTCTCG |
| 881 | GGAGGCAGGC ACCGAGAAGA TCAAGGGCCT GAACGAGGTG |
| 921 | CTGAATCTGG CCATCCAGAA GAATGATGAG ACAGCCCACA |
| 961 | TCATCGCCTC CCTGCCACAC AGATTCATCC CCCTGTTTAA |
| 1001 | GCAGATCCTG TCCGATAGGA ACACCCTGTC TTTCATCCTG |
| 1041 | GAGGAGTTTA AGAGCGACGA GGAAGTGATC CAGTCCTTCT |
| 1081 | GCAAGTACAA GACACTGCTG AGAAACGAGA ACGTGCTGGA |
| 1121 | GACAGCCGAG GCCCTGTTTA ACGAGCTGAA CAGCATCGAC |
| 1161 | CTGACACACA TCTTCATCAG CCACAAGAAG CTGGAGACAA |
| 1201 | TCAGCAGCGC CCTGTGCGAC CACTGGGATA CACTGAGGAA |
| 1241 | TGCCCTGTAT GAGCGGAGAA TCTCCGAGCT GACAGGCAAG |
| 1281 | ATCACCAAGT CTGCCAAGGA GAAGGTGCAG CGCAGCCTGA |
| 1321 | AGCACGAGGA TATCAACCTG CAGGAGATCA TCTCTGCCGC |
| 1361 | AGGCAAGGAG CTGAGCGAGG CCTTCAAGCA GAAAACCAGC |
| 1401 | GAGATCCTGT CCCACGCACA CGCCGCCCTG GATCAGCCAC |
| 1441 | TGCCTACAAC CCTGAAGAAG CAGGAGGAGA AGGAGATCCT |
| 1481 | GAAGTCTCAG CTGGACAGCC TGCTGGGCCT GTACCACCTG |
| 1521 | CTGGACTGGT TTGCCGTGGA TGAGTCCAAC GAGGTGGACC |
| 1561 | CCGAGTTCTC TGCCCGGCTG ACCGGCATCA AGCTGGAGAT |
| 1601 | GGAGCCTTCT CTGAGCTTCT ACAACAAGGC CAGAAATTAT |
| 1641 | GCCACCAAGA AGCCCTACTC CGTGGAGAAG TTCAAGCTGA |
| 1681 | ACTTTCAGAT GCCTACACTG GCCTCTGGCT GGGACGTGAA |
| 1721 | TAAGGAGAAG AACAATGGCG CCATCCTGTT TGTGAAGAAC |
| 1761 | GGCCTGTACT ATCTGGGCAT CATGCCAAAG CAGAAGGGCA |
| 1801 | GGTATAAGGC CCTGAGCTTC GAGCCCACAG AGAAAACCAG |
| 1841 | CGAGGGCTTT GATAAGATGT ACTATGACTA CTTCCCTGAT |
| 1881 | GCCGCCAAGA TGATCCCAAA GTGCAGCACC CAGCTGAAGG |
| 1921 | CCGTGACAGC CCACTTTCAG ACCCACACAA CCCCCATCCT |
| 1961 | GCTGTCCAAC AATTTCATCG AGCCTCTGGA GATCACAAAG |
| 2001 | GAGATCTACG ACCTGAACAA TCCTGAGAAG GAGCCAAAGA |
| 2041 | AGTTTCAGAC AGCCTACGCC AAGAAAACCG GCGACCAGAA |
| 2081 | GGGCTACAGA GAGGCCCTGT GCAAGTGGAT CGACTTCACA |
| 2121 | AGGGATTTTC TGTCCAAGTA TACCAAGACA ACCTCTATCG |
| 2161 | ATCTGTCTAG CCTGCGGCCA TCCTCTCAGT ATAAGGACCT |
| 2201 | GGGCGAGTAC TATGCCGAGC TGAATCCCCT GCTGTACCAC |
| 2241 | ATCAGCTTCC AGAGAATCGC CGAGAAGGAG ATCATGGATG |
| 2281 | CCGTGGAGAC AGGCAAGCTG TACCTGTTCC AGATCTATAA |
| 2321 | CAAGGACTTT GCCAAGGGCC ACCACGGCAA GCCTAATCTG |
| 2361 | CACACACTGT ATTGGACCGG CCTGTTTTCT CCAGAGAACC |
| 2401 | TGGCCAAGAC AAGCATCAAG CTGAATGGCC AGGCCGAGCT |
| 2441 | GTTCTACCGC CCTAAGTCCA GGATGAAGAG GATGGCACAC |
| 2481 | CGGCTGGGAG AGAAGATGCT GAACAAGAAG CTGAAGGATC |
| 2521 | AGAAAACCCC AATCCCCGAC ACCCTGTACC AGGAGCTGTA |
| 2561 | CGACTATGTG AATCACAGAC TGTCCCACGA CCTGTCTGAT |
| 2601 | GAGGCCAGGG CCCTGCTGCC CAACGTGATC ACCAAGGAGG |
| 2641 | TGTCTCACGA GATCATCAAG GATAGGCGCT TTACCAGCGA |
| 2681 | CAAGTTCTTT TTCCACGTGC CTATCACACT GAACTATCAG |
| 2721 | GCCGCCAATT CCCCATCTAA GTTCAACCAG AGGGTGAATG |
| 2761 | CCTACCTGAA GGAGCACCCC GAGACACCTA TCATCGGCAT |
| 2801 | CGATCGGGGC GAGAGAAACC TGATCTATAT CACAGTGATC |
| 2841 | GCCTCCACCG GCAAGATCCT GGAGCAGCGG AGCCTGAACA |
| 2881 | CCATCCAGCA GTTTGATTAC CAGAAGAAGC TGGACAACAG |
| 2921 | GGAGAAGGAG AGGGTGGCAG CAAGGCAGGC CTGGTCTGTG |
| 2961 | GTGGGCACAA TCAAGGATCT GAAGCAGGGC TATCTGAGCC |
| 3001 | AGGTCATCCA CGAGATCGTG GACCTGATGA TCCACTACCA |
| 3041 | GGCCGTGGTG GTGCTGGAGA ACCTGAATTT CGGCTTTAAG |
| 3081 | AGCAAGAGGA CCGGCATCGC CGCGAAGGCC GTGTACCAGC |
| 3121 | AGTTCGAGAA GATGCTGATC GATAAGCTGA ATTGCCTGGT |
| 3161 | GCTGAAGGAC TATCCAGCAG AGAAAGTGGG AGGCGTGCTG |
| 3201 | AACCCATACC AGCTGACAGA CCAGTTCACC TCCTTTGCCA |
| 3241 | AGATGGGCAC CCAGTCTGGC TTCCTGTTTT ACGTGCCTGC |
| 3281 | CCCATATACA TCTAAGATCG ATCCCCTGAC CGGCTTCGTG |
| 3321 | GACCCCTTCG TGTGGAAAAC CATCAAGAAT CACGAGAGCC |
| 3361 | GCAAGCACTT CCTGGAGGGC TTCGACTTTC TGCACTACGA |
| 3401 | CGTGAAAACC GGCGACTTCA TCCTGCACTT TAAGATGAAC |
| 3441 | AGAAATCTGT CCTTCCAGAG GGGCCTGCCC GGCTTTATGC |
| 3481 | CTGCATGGGA TATCGTGTTC GAGAAGAACG AGACACAGTT |
| 3521 | TGACGCCAAG GGCACCCCTT TCATCGCCGG CAAGAGAATC |
| 3561 | GTGCCAGTGA TCGAGAATCA CAGATTCACC GGCAGATACC |
| 3601 | GGGACCTGTA TCCTGCCAAC GAGCTGATCG CCCTGCTGGA |
| 3641 | GGAGAAGGGC ATCGTGTTCA GGGATGGCTC CAACATCCTG |
| 3681 | CCAAAGCTGC TGGAGAATGA CGATTCTCAC GCCATCGACA |
| 3721 | CCATGGTGGC CCTGATCCGC AGCGTGCTGC AGATGCGGAA |
| 3761 | CTCCAATGCC GCCACAGGCG AGGACTATAT CAACAGCCCC |
| 3801 | GTGCGCGATC TGAATGGCGT GTGCTTCGAC TCCCGGTTTC |
| 3841 | AGAACCCAGA GTGGCCCATG GACGCCGATG CCAATGGCGC |
| 3881 | CTACCACATC GCCCTGAAGG GCCAGCTGCT GCTGAATCAC |
| 3921 | CTGAAGGAGA GCAAGGATCT GAAGCTGCAG AACGGCATCT |
| 3961 | CCAATCAGGA CTGGCTGGCC TACATCCAGG AGCTGCGCAA |
| 4001 | C |

### Guide RNA Delivery

There are different ways to deliver guide RNAs and corrective nucleases. The first and probably the most straightforward approach is to use a vector-based CRISPR-Cas9 system encoding the corrective nuclease and guide RNA (e.g., sgRNA) from the same vector, thus avoiding multiple transfections of different components. The second is to deliver the mixture of the Cas9 mRNA and the sgRNA, and the third strategy is to deliver the mixture of the Cas9 protein and the sgRNA.

In some cases, the guide RNAs can be delivered to cells or administered to subjects in the form of an expression cassette or vector that can express one or more of the guide RNAs. Corrective nucleases can also be delivered to cells or administered to the subjects in the form of an expression cassette or vector that can express one or more corrective nucleases. The corrective nucleases can also be combined with their respective gRNAs and delivered as RNA-protein complexes (RNPs). Hence, the RNPs can be pre-assembled outside of the cell and introduced into the cell.

Hence, the guide RNAs can be recombinantly expressed in the cells. The corrective nuclease can also be expressed in the same cell with one or more gRNAs. The guide RNAs and corrective nucleases can be introduced in form of nucleic acid molecules encoding the guide RNAs and/or corrective nucleases. The nucleic acid molecules encoding the guide RNAs and/or corrective nuclease proteins can be provided in expression cassettes or expression vectors.

The expression cassettes can be within vectors. Vectors can, for example, be expression vectors such as viruses or other vectors that are readily taken up by the cells. Examples of vectors that can be used include, for example, adeno-associated virus (AAV) gene transfer vectors, lentiviral vectors, retroviral vectors, virus-like particles (VLPs), herpes virus vectors, e.g., cytomegalovirus vectors, herpes simplex virus vectors, varicella zoster virus vectors, adenovirus vectors, e.g., helper-dependent adenovirus vectors, adenovirus-AAV hybrids, rabies virus vectors, vesicular stomatitis virus (VSV) vectors, coronavirus vectors, poxvirus vectors and the like. Non-viral vectors may be employed to deliver the expression vectors, e.g., liposomes, nanoparticles, microparticles, lipoplexes, polyplexes, nanotubes, and the like. In one embodiment, two or more expression vectors are administered, for instance, each encoding a distinct guide RNA, a distinct corrective nuclease, or a combination thereof.

The expression cassettes or expression vectors include promoter sequences that are operably linked to the nucleic acid segment encoding the guide RNAs, corrective nucleases, or combinations thereof. Methods for ensuring expression of a functional guide RNA, corrective nuclease or combinations thereof can involve expression from a transgene, expression cassette, or expression vector. For example, the nucleic acid segments encoding the selected guide RNAs, or combinations thereof can be present in a vector, such as a plasmid, cosmid, virus, bacteriophage or another vector available for genetic engineering. The coding sequences inserted in the vector can be synthesized by standard methods or isolated from natural sources. The coding sequences may further be ligated to transcriptional regulatory elements, termination sequences, and/or to other amino acid encoding sequences. Such regulatory sequences can provide initiation of transcription, internal ribosomal entry sites (IRES) (Owens, Proc. Natl. Acad. Sci. USA 98:1471-1476 (2001)) and optionally regulatory elements ensuring termination of transcription and stabilization of the transcript. Non-limiting examples for regulatory elements ensuring the initiation of transcription comprise a translation initiation codon, transcriptional enhancers such as e.g. the SV40-enhancer, insulators and/or promoters. The promoter can be a constitutive promoter, and inducible promoter, or a tissue-specific promoter. Examples of promoters that can be used include the cytomegalovirus (CMV) promoter, SV40-promoter, RSV-promoter (Rous sarcoma virus), the lacZ promoter, chicken beta-actin promoter, CAG-promoter (a combination of chicken beta-actin promoter and cytomegalovirus immediate-early enhancer), the gai 10 promoter, human elongation factor 1α-promoter, AOX1 promoter, GALI promoter CaM-kinase promoter, the lac, trp or tac promoter, the lacUV5 promoter, the Autographa californica multiple nuclear polyhedrosis virus (AcMNPV) polyhedral promoter, or a globin intron in mammalian and other animal cells. Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing. Moreover, elements such as origin of replication, drug resistance gene or regulators (as part of an inducible promoter) may also be included.

The expression cassettes and/or expression vectors can be introduced into cells. The cells can be any mammalian or avian cell. For example, the cells can be human cells, or cells from a domesticated animal, a zoo animal, or an experimental animal. The cells can be obtained from a subject in need of treatment. The cells can be autologous or allogenic cells relative to a subject. In some cases, the cells can be stem cells, induced pluripotent stem cells, cardiac progenitor cells, and/or cardiomyocytes. The allogenic cells can be typed to match those of a subject.

The guide RNAs can also be introduced into cells or administered to subjects in the form of Ribonucleoproteins (RNPs). The corrective nuclease can be pre-bound with their respective gRNAs prior to introduction into cells. The advantage of RNP delivery of Cas-gRNA complexes is that complex formation is readily controlled ex vivo and the selected Cas polypeptides can independently be complexed with selected guide RNA sequences so that the structure and compositions of the desired complexes is known with certainty. These RNPs are quite stable, with no apparent exchange of gRNAs. Hence, the nuclease-gRNA RNP can carry a selected gRNA to the site of genomic editing.

For example, Cas RNP can be prepared by incubating the Cas proteins with the selected gRNA using a molar excess of gRNA relative to protein (e.g., using about a 1:1.1 to 1:1.4 protein to gRNA molar ratio). The buffer to be used during such incubation can include 20 mM HEPES (pH 7.5), 150 mM KCl, 1 mM MgCl₂, 10% glycerol and 1 mM TCEP. Incubation can be done at 37 °C for about 5 minutes to about 30 minutes (usually 10 minutes is sufficient). When reference DNA or an HDR template is used, it can be added to the Cas RNP.

Nucleofection can be employed to introduce the Cas RNP into cells. See Lin et al., Enhanced homology-directed human genome engineering by controlled timing of CRISPR/Cas9 delivery. Elife 3:e04766. For example, nucleofection reactions can involve mixing approximately 1×10⁴ to 1×10⁷ cells in about 10 μl to 40 μl of nucleofection reagent with about 5μ to 30 μl of RNP: DNA. In some instances, about 2×10⁵ cells are mixed with about 20 μl of nucleofection reagent and about 10 μl RNP: DNA. After electroporation, growth media is added, and the cells are transferred to tissue culture plates for growth and evaluation. The nucleofection reagents and machines are available from Lonza (Allendale, NJ).

Thus, the invention provides compounds for use in medical therapy, such as gene therapy vectors that inhibit or prevent cardiomyopathy.

### Cell Therapies

In some cases, cells can be modified in vitro and then administered to a subject. For example, cells can be contacted and/or treated with any of the guide RNAs, ribonucleoprotein complexes, expression cassettes/expression vectors or modifying agents described herein. Such modification can reduce MYH7 expression from a MYH7 allele that expresses a MYH7 protein correlated with a cardiomyopathy condition or disease. The cells can be autologous or allogeneic to the subject so administered. For example, the cells can be obtained from a subject, then these cells can be contacted and/or treated with any of the guide RNAs, ribonucleoprotein complexes, expression cassettes/expression vectors or modifying agents described herein to generate modified cells. The cells to be modified can, for example, be cardiomyocytes, epithelial cells, or neuronal cells.

The modified cells can be expanded in culture to form a population of modified cells, and the population of cells can be administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a device or a vehicle so that a population of liposomes, exosomes or microvesicles.

Cells are administered to patients at various time points, for example, as immunotherapy, or to retard or inhibit tumor growth. Administration of cells should improve the cardiomyopathie condition of the subject. Treatment may comprise the cells administered alone or with any of the guide RNAs, ribonucleoprotein complexes, expression cassettes/expression vectors or modifying agents described herein. Such agents can be administered separately from or with the modified cells. For example, the modified cells may be administered prior to, during, or after administering any of the guide RNAs, ribonucleoprotein complexes, expression cassettes/expression vectors or modifying agents described herein.

### Administration

Guide RNAs, expression cassettes/expression vectors that can express the guide RNA, modified cells, or combinations thereof can be administered to subjects. Cells that have been modified to eliminate problematic MYH7 mutations can be administered to subjects with or without the guide RNAs, ribonucleoprotein complexes, expression cassettes/expression vectors or modifying agents described herein. Such guide RNAs, expression cassettes, expression vectors, and cells generated as described herein can be employed for tissue reconstitution or regeneration in a human patient or other subjects. Patients or subjects can be in need of such treatment. In some cases, the patients or subjects may not yet exhibit any symptoms of disease or a medical condition. However, a patient or subject to be treated may have at least one MYH7 allele correlated with development or existence of a cardiomyopathy.

The guide RNAs, expression cassettes, expression vectors, and cells are administered in a manner that permits them to be incorporated into, graft or migrate to a specific tissue site, such as into cardiomyocytes or cardiac tissues. Such guide RNAs, expression cassettes, expression vectors, and cells can reconstitute or regenerate functionally deficient areas of tissues, including cardiomyocytes or cardiac tissues. Devices are available that can be adapted for administering cells, for example, to cardiomyocytes or cardiac tissues.

For therapy, guide RNAs, expression cassettes, expression vectors, and/or cardiac cells can be administered locally or systemically. Administration can be by injection, catheter, implantable device, or the like. The guide RNAs, expression cassettes, expression vectors, and cells can be administered in any physiologically acceptable excipient or carrier that does not adversely affect the subject. For example, the guide RNAs, expression cassettes, expression vectors, and cells can be administered, for example, intravenously. Methods of administering the guide RNAs, expression cassettes, expression vectors, and/or cells to subjects, particularly human subjects, include injection or implantation of the guide RNAs, expression cassettes, expression vectors, and cells into target sites or they can be inserted into a delivery device which facilitates introduction, uptake, incorporation, or implantation of the expression cassettes, expression vectors, and cells. Such delivery devices include tubes, e.g., catheters, for introducing cells, expression vectors, and fluids into the body of a recipient subject. The tubes can additionally include a needle, e.g., a syringe, through which the cells of the invention can be introduced into the subject at a desired location. Multiple injections may be made using this procedure.

As used herein, the term “solution” includes a carrier or diluent in which the guide RNAs, expression cassettes, expression vectors, and cells of the invention remain viable and/or functional. Carriers and diluents that can be used include saline, aqueous buffer solutions, solvents and/or dispersion media. The use of such carriers and diluents are available in the art. The solution can be sterile and fluid to the extent that easy syringe-ability exists.

The guide RNAs, expression cassettes, expression vectors, and cells can also be embedded in a support matrix. Suitable ingredients include matrix proteins that support or promote the incorporation of adhesion of the guide RNAs, expression cassettes, expression vectors, and modified cells. In another embodiment, the composition may include physiologically acceptable matrix scaffolds. Such physiologically acceptable matrix scaffolds can be resorbable and/or biodegradable.

In some cases, cardiac cells, such as cardiomyocytes can be modified to express the guide RNAs and optionally the corrective nuclease. In addition, cardiac cells can be modified by the guide RNAs and corrective nucleases to generate a population of modified cells that do not have a pathological mutation in their MYH7 gene.

A population of modified cells generated by the methods described herein can include low percentages of non-cardiac cells (e.g., fibroblasts and/or endothelial cells). For example, a population of modified cells for use in compositions and for administration to subjects can have less than about 90% non-cardiac cells, less than about 85% non-cardiac cells, less than about 80% non-cardiac cells, less than about 75% non-cardiac cells, less than about 70% non-cardiac cells, less than about 65% non-cardiac cells, less than about 60% non-cardiac cells, less than about 55% non-cardiac cells, less than about 50% non-cardiac cells, less than about 45% non-cardiac cells, less than about 40% non-cardiac cells, less than about 35% non-cardiac cells, less than about 30% non-cardiac cells, less than about 25% non-cardiac cells, less than about 20% non-cardiac cells, less than about 15 non-cardiac cells, less than about 12% non-cardiac cells, less than about 10% non-cardiac cells, less than about 8% non-cardiac cells, less than about 6% non-cardiac cells, less than about 5% non-cardiac cells, less than about 4% non-cardiac cells, less than about 3% non-cardiac cells, less than about 2% non-cardiac cells, or less than about 1% non-cardiac cells of the total cells in the cell population.

Many cell types are capable of migrating to an appropriate site for regeneration and differentiation within a subject. To determine the suitability of various therapeutic administration regimens and dosages of cell compositions, the cells can first be tested in a suitable animal model. At one level, cells are assessed for their ability to survive and maintain their phenotype in vivo. Cells can also be assessed to ascertain whether they migrate to diseased or injured sites in vivo, or to determine an appropriate number, or dosage, of cells to be administered. Cell compositions can be administered to immunodeficient animals (such as nude mice, or animals rendered immunodeficient chemically or by irradiation). Tissues can be harvested after a period of regrowth and assessed as to whether the administered cells or progeny thereof are still present, are alive, and/or have migrated to desired or undesired locations.

Injected cells can be traced by a variety of methods. For example, cells containing or expressing a detectable label (such as green fluorescent protein, or beta-galactosidase) can readily be detected. The cells can be pre-labeled, for example, with BrdU or [³H]-thymidine, or by introduction of an expression cassette that can express green fluorescent protein, or beta-galactosidase. Alternatively, the modified cells can be detected by their expression of a cell marker that is not expressed by the animal employed for testing (for example, a human-specific antigen when injecting cells into an experimental animal). The presence and phenotype of the administered population of modified cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for RNA indicative of a cardiac phenotype.

Modified cells can be included in the compositions in varying amounts depending upon the extent of disease or the condition of the subject. For example, the compositions can be prepared in liquid form for local or systemic administration containing about 10³ to about 10¹² modified cells, or about 10⁴ to about 10¹⁰ modified cells, or about 10⁵ to about 10⁸ modified cells.

One or more RNPs containing a guide RNA or expression vectors that can express one or more guide RNAs, corrective nuclease, or a combination thereof can also be administered with or without the cells.

The guide RNA, corrective nuclease, and/or RNP with or without additional cells may be administered in a composition as a single dose, in multiple doses, in a continuous or intermittent manner, depending, for example, upon the recipient's physiological condition, whether the purpose of the administration is in response to traumatic injury or for more sustained therapeutic purposes, and other factors known to skilled practitioners. The administration of the compositions of the invention may be as a single dose, or essentially continuous over a preselected period of time, or it may be in a series of spaced doses. Both local and systemic administration is contemplated.

It will be appreciated that the amounts of guide RNAs, corrective nucleases, RNPs, and/or cells for use in treatment will vary not only with the particular carrier selected but also with the route of administration, the nature of the condition being treated and the age and condition of the patient. Ultimately, the attendant health care provider may determine proper dosage.

### Embodiments

In one embodiment, a composition is provided comprising (a) a carrier; (b) a guide RNA comprising an RNA sequence having at least 95% sequence identity to a sequence selected from the group consisting of SEQ ID NOs: 1-6, 13-24 and 25; and (c) a Cas nuclease. The guide RNA directs the Cas nuclease to a genomic site within a protein-coding exon of a human MYH7 gene comprising a heterozygous single nucleotide polymorphism (SNP). In some embodiments, the heterozygous SNP is selected from the group consisting of rs2069540, rs735711, rs7157716, rs2231126, and rs2069542. The guide RNA is configured to recognize one allele at the SNP site with selectivity over the other allele, as demonstrated in iPSC-derived cardiomyocytes (FIG. 1) and iPSCs (FIG. 2).

In some embodiments, the guide RNA of the composition comprises an RNA sequence selected from the group consisting of SEQ ID NOs: 14, 17, 18, 19, 22, 23, 24 and 25. For example, the guide RNA may comprise the sequence of SEQ ID NO: 14, which targets the alternate allele of rs2231126, as shown in FIG. 2 (SPg004) and FIG. 3A (ABE VLP Spg004). In another example, the guide RNA may comprise the sequence of SEQ ID NO: 25, which targets rs2069540, as shown in FIG. 3A (ABE VLP Spg032), FIG. 3B (Cas9 VLP Spg032), and FIG. 3C (Cas9 VLP Spg004 dose curve). In another example, the guide RNA may comprise the sequence of SEQ ID NO: 22, which targets the alternate allele of rs2069542, as shown in FIG. 2 (SPg029). In yet another example, the guide RNA may comprise the sequence of SEQ ID NO: 19, which targets the reference allele of rs2069542, as shown in FIG. 2 (SPg026) and FIG. 3B (Cas9 VLP Spg026).

In some embodiments, the Cas nuclease of the composition is selected from the group consisting of Streptococcus pyogenes Cas9 (SpCas9), Staphylococcus aureus Cas9 (SaCas9), Francisella novicida Cas12a (FnCas12a), Neisseria meningitidis Cas9 (Nme2Cas9), and combinations thereof. For example, SpCas9 may be used as the Cas nuclease, as demonstrated in the editing experiments shown in FIGS. 1, 2, 3B, and 3C.

In some embodiments, the guide RNA and the Cas nuclease of the composition are complexed as a ribonucleoprotein (RNP). The RNP may be pre-assembled outside of the cell prior to delivery. In some embodiments, the Cas nuclease is pre-bound with the guide RNA at a molar ratio of about 1:1.1 to about 1:1.4 protein to guide RNA.

In some embodiments, the carrier of the composition comprises a lipid nanoparticle (LNP), a virus-like particle (VLP), or an adeno-associated virus (AAV) vector. For example, VLPs may be used to deliver the guide RNA and Cas nuclease to iPSC-derived cardiomyocytes, as demonstrated in FIGS. 3A, 3B, and 3C, where both ABE VLPs and Cas9 VLPs were used to deliver guide RNAs Spg004, Spg032, Spg026, and Spg029 to cardiomyocytes.

In some embodiments, the composition further comprises a second guide RNA comprising an RNA sequence having at least 95% sequence identity to a sequence selected from the group consisting of SEQ ID NOs: 1-6, 13-24 and 25, wherein the second guide RNA targets a different SNP allele than the first guide RNA. For example, the composition may comprise a first guide RNA targeting the alternate allele of a given SNP and a second guide RNA targeting the reference allele of the same SNP, or the composition may comprise guide RNAs targeting alleles at two different SNP sites. As shown in FIG. 1, guide RNAs targeting the alternate allele (Spg022) and the reference allele (Spg023) of rs2069540 each demonstrate allele-specific editing in iPSC-derived cardiomyocytes. Similarly, guide RNAs targeting the alternate allele (Spg008) and the reference allele (Spg018) of rs735711 each demonstrate allele-specific editing. Such paired guide RNAs may be included in a single composition to provide coverage for patients carrying the disease mutation in cis with either allele of a given SNP.

In some embodiments, the composition comprises an expression vector comprising a first promoter operably linked to a nucleic acid encoding the guide RNA and a second promoter operably linked to a nucleic acid encoding the Cas nuclease. The expression vector may be an adeno-associated virus (AAV) vector, a lentiviral vector, or a plasmid vector. The first and second promoters may be the same or different.

In some embodiments, at least one of the first promoter and the second promoter is a cardiac-specific promoter. Use of a cardiac-specific promoter restricts expression of the guide RNA, the Cas nuclease, or both to cardiac cells, thereby reducing off-target expression in non-cardiac tissues. Examples of cardiac-specific promoters include, but are not limited to, the cardiac troponin T (cTnT) promoter, the alpha-myosin heavy chain (α-MHC) promoter, and the myosin light chain 2v (MLC2v) promoter.

In one embodiment, a method of editing an endogenous MYH7 gene in a cell is provided. The method comprises contacting the cell with (i) a guide RNA comprising an RNA sequence having at least 95% sequence identity to a sequence selected from the group consisting of SEQ ID NOs: 1-6, 13-24 and 25, and (ii) a Cas nuclease. The guide RNA directs the Cas nuclease to cleave one allele of the MYH7 gene at a genomic site comprising a heterozygous single nucleotide polymorphism (SNP), thereby introducing an insertion or deletion that generates a premature termination codon in the cleaved allele. Expression of a mutant MYH7 protein encoded by the cleaved allele is reduced relative to expression prior to the contacting. As shown in FIGS. 1 and 2, guide RNAs targeting SNPs rs2069540, rs735711, rs2231126, and rs2069542 introduce allele-specific modifications in iPSC-derived cardiomyocytes and iPSCs. As shown in FIGS. 3A and 3B, allelic RNA expression analysis confirms that target allele expression is reduced following delivery of ABE VLPs and Cas9 VLPs carrying the guide RNAs.

In some embodiments, the premature termination codon generated by the insertion or deletion instigates nonsense-mediated decay (NMD) of an mRNA transcript from the cleaved allele. The NMD pathway recognizes and degrades the mRNA transcript containing the premature termination codon, thereby reducing or eliminating expression of the mutant MYH7 protein. As shown in the allelic expression panels of FIGS. 3A and 3B, target allele expression measured by cDNA-based next-generation sequencing is reduced following guide RNA delivery, consistent with NMD-mediated degradation of the edited transcript.

In some embodiments, the cell is heterozygous for a dominant mutation in the MYH7 gene, and the SNP targeted by the guide RNA is in cis with the dominant mutation on the same allele. In such embodiments, the guide RNA directs the Cas nuclease to cleave the allele carrying both the SNP and the dominant mutation, while the wild-type allele, which does not carry the targeted SNP variant, remains unedited and continues to express functional beta-myosin heavy chain protein.

In some embodiments, the guide RNA and the Cas nuclease are delivered to the cell as a ribonucleoprotein (RNP) complex. In some embodiments, the RNP is pre-assembled by incubating the Cas nuclease with the guide RNA prior to delivery to the cell. The RNP may be introduced into the cell by nucleofection, electroporation, or other transfection methods.

In some embodiments, the guide RNA and the Cas nuclease are delivered to the cell via a virus-like particle (VLP). As demonstrated in FIGS. 3A, 3B, and 3C, VLPs carrying ABE or Cas9 together with allele-specific guide RNAs (Spg004, Spg032, Spg026, Spg029) achieve allele-specific genome editing in iPSC-derived cardiomyocytes. VLP delivery provides transient exposure of the cell to the editing reagents, which may reduce the duration of nuclease activity and limit off-target editing.

In some embodiments, the guide RNA used in the editing method comprises an RNA sequence selected from the group consisting of SEQ ID NOs: 14, 17, 18, 19, 22, 23, 24 and 25. Each of these guide RNA sequences has been demonstrated to exhibit allele-specific editing activity at its respective target SNP, as shown in FIGS. 1, 2, 3A, 3B, and 3C.

In some embodiments, the cell is an iPSC-derived cardiomyocyte or a cardiomyocyte. As shown in FIG. 1, guide RNAs Spg022, Spg023, Spg008, and Spg018 achieve allele-specific editing in iPSC-derived cardiomyocytes. As shown in FIGS. 3A, 3B, and 3C, guide RNAs Spg004, Spg032, Spg026, and Spg029 achieve allele-specific editing in iPSC-derived cardiomyocytes when delivered via VLPs. The cell may also be a primary cardiomyocyte obtained from a subject or a cardiomyocyte differentiated from a stem cell or progenitor cell.

In some embodiments, the Cas nuclease used in the editing method is SpCas9 or an adenosine base editor (ABE) comprising a Cas9 nickase domain. As shown in FIG. 3A, ABE VLPs carrying Spg004 and Spg032 achieve allele-specific base editing at the genomic DNA level and corresponding reduction in target allele expression at the RNA level in iPSC-derived cardiomyocytes. As shown in FIGS. 3B and 3C, Cas9 VLPs carrying Spg032, Spg026, Spg004, and Spg029 achieve allele-specific indel formation in iPSC-derived cardiomyocytes. An ABE comprising a Cas9 nickase domain introduces a targeted base conversion (e.g., A-to-G) rather than a double-strand break, which may reduce the frequency of undesired indel byproducts at the target site.

In one embodiment, a method of treating a subject having a heterozygous dominant mutation in the MYH7 gene correlated with cardiomyopathy is provided. The method comprises administering to the subject (i) a guide RNA comprising an RNA sequence having at least 95% sequence identity to a sequence selected from the group consisting of SEQ ID NOs: 1-6, 13-24 and 25, and (ii) a Cas nuclease. The guide RNA directs the Cas nuclease to a genomic site within a protein-coding exon of the MYH7 gene comprising a heterozygous single nucleotide polymorphism (SNP). The SNP is in cis with the dominant mutation. The administering reduces expression of a mutant MYH7 protein from the allele carrying the dominant mutation in cardiac cells of the subject. Because MYH7 is haplosufficient, expression from the remaining wild-type allele is sufficient to support normal cardiac function.

In some embodiments, the guide RNA and the Cas nuclease are administered to the subject as a ribonucleoprotein complex within a virus-like particle (VLP) or a lipid nanoparticle (LNP). VLP-mediated delivery of Cas9 and ABE ribonucleoprotein complexes to cardiomyocytes has been demonstrated in iPSC-derived cardiomyocytes, as shown in FIGS. 3A, 3B, and 3C. LNP-mediated delivery may be used to deliver mRNA encoding the Cas nuclease together with the guide RNA to cardiac cells in vivo. The VLP or LNP may be formulated for systemic or local administration.

In some embodiments, the guide RNA administered to the subject comprises an RNA sequence selected from the group consisting of SEQ ID NOs: 14, 17, 18, 19, 22, 23, 24 and 25. The selection of the guide RNA for a given subject may depend on the SNP genotype of the subject. For example, a subject who is heterozygous at rs2231126 and carries a disease-causing MYH7 mutation in cis with the alternate allele of rs2231126 may be treated with a guide RNA comprising the sequence of SEQ ID NO: 14 (Spg004). A subject who is heterozygous at rs2069542 and carries a disease-causing MYH7 mutation in cis with the alternate allele of rs2069542 may be treated with a guide RNA comprising the sequence of SEQ ID NO: 22 (Spg029). A subject who is heterozygous at rs2069540 and carries a disease-causing MYH7 mutation in cis with the reference allele of rs2069540 may be treated with a guide RNA comprising the sequence of SEQ ID NO: 25 (Spg032).

In some embodiments, the administering comprises delivering the guide RNA and the Cas nuclease to cardiac tissue of the subject, and expression from a wild-type MYH7 allele in the cardiac tissue is maintained. The guide RNA and Cas nuclease may be delivered locally to cardiac tissue by injection, catheter-based delivery, or implantable device. In some embodiments, the guide RNA and Cas nuclease are delivered systemically and targeted to cardiac tissue through the use of cardiac-tropic delivery vehicles, cardiac-specific promoters, or both. As shown in the allelic expression panels of FIGS. 3A and 3B, non-target allele expression is maintained following delivery of allele-specific guide RNAs, confirming that the wild-type allele continues to be expressed after editing of the target allele. Maintenance of wild-type MYH7 expression is consistent with the haplosufficiency of MYH7 and supports the therapeutic utility of allele-specific gene silencing for treating dominant-negative MYH7 cardiomyopathy.

In some embodiments, the subject has been genotyped to confirm that the subject is heterozygous at the targeted SNP and that the disease-causing MYH7 mutation is in cis with the allele targeted by the guide RNA. Genotyping may be performed by sequencing, allele-specific PCR, SNP array, or other methods available in the art. In some embodiments, the method further comprises confirming, after administration, that expression of the mutant MYH7 protein is reduced or eliminated in cardiac cells of the subject.

In some embodiments, the cardiomyopathy correlated with the heterozygous dominant mutation in the MYH7 gene is hypertrophic cardiomyopathy (HCM), dilated cardiomyopathy (DCM), or restrictive cardiomyopathy (RCM). Over 200 different mutations in MYH7 have been associated with cardiomyopathy. Because the method targets a common heterozygous SNP rather than the disease-causing mutation itself, the method is applicable to subjects carrying any of these mutations, provided the mutation is in cis with the targeted SNP allele.

In some embodiments, the method comprises administering the guide RNA and the Cas nuclease at a dose and by a route sufficient to edit a therapeutically effective proportion of cardiomyocytes in the subject. The guide RNA and Cas nuclease may be administered in a single dose or in multiple doses. As shown in FIG. 3C, dose-dependent allele-specific editing is observed in iPSC-derived cardiomyocytes treated with increasing doses of Cas9 VLPs carrying Spg004 and Spg029, indicating that the level of allele-specific editing can be modulated by adjusting the dose of the delivered reagents.
1. In one embodiment, a guide RNA is provided comprising an RNA sequence having at least 80%, at least 85%, at least 90%, at least 93%, at least 95%, at least 96%, at least 97%, or at least 98% sequence identity to any of SEQ ID NOs: 1-6 or 13-24, such as SEQ ID NOs: 14, 17, 18, 19, 22, 23, or 24.
2. In some embodiments, each of the guide RNAs includes a Protospacer Adjacent Motif (PAM) sequence.
3. In some embodiments, a cell or population of cells is provided comprising the guide RNA described herein.
4. In some embodiments, a composition is provided comprising a carrier and one or more guide RNAs with an RNA sequence having at least 80%, at least 85%, at least 90%, at least 93%, at least 95%, at least 96%, at least 97%, or at least 98% sequence identity to any of SEQ ID NOs: 1-6 or 13-24, such as SEQ ID NOs: 14, 17, 18, 19, 22, 23, or 24.
5. In some embodiments, the composition further comprises a nuclease.
6. In some embodiments, the nuclease is a Streptococcus pyogenes Cas9 (SpCas9), Staphylococcus aureus Cas9 (SaCas9), a Francisella novicida Cas12a, Neisseria meningitidis Cas9 (Nme2Cas9), or a combination thereof.
7. In some embodiments, a cell or population of cells is provided comprising the composition described herein.
8. In some embodiments, the cell or population of cells are cardiac cells.
9. In some embodiments, a ribonucleoprotein complex is provided comprising at least one nuclease and at least one guide RNA comprising an RNA sequence having at least 80%, at least 85%, at least 90%, at least 93%, at least 95%, at least 96%, at least 97%, or at least 98% sequence identity to any of SEQ ID NOs: 1-6 or 13-24, such as SEQ ID NOs: 14, 17, 18, 19, 22, 23, or 24.
10. In some embodiments, each of the guide RNAs in the ribonucleoprotein complex includes a Protospacer Adjacent Motif (PAM) sequence.
11. In some embodiments, the nuclease of the ribonucleoprotein complex is a Cas9 nuclease.
12. In some embodiments, the nuclease of the ribonucleoprotein complex is a Streptococcus pyogenes Cas9 (SpCas9), Staphylococcus aureus Cas9 (SaCas9), a Francisella novicida Cas12a, Neisseria meningitidis Cas9 (Nme2Cas9), or a combination thereof.
13. In some embodiments, an expression system is provided comprising one or more expression cassettes or expression vectors comprising a first promoter operably linked to a nucleic acid segment with at least 80%, at least 85%, at least 90%, at least 93%, at least 95%, at least 96%, at least 97%, or at least 98% sequence identity to any of SEQ ID NOs: 1-6 or 13-24, such as SEQ ID NOs: 14, 17, 18, 19, 22, 23, or 24.
14. In some embodiments, the first promoter is an inducible promoter, a tissue-specific promoter, or a cell-type specific promoter.
15. In some embodiments, the expression system further comprises an expression cassette or expression vector comprising a second promoter operably linked to a nucleic acid segment encoding a nuclease.
16. In some embodiments, the nuclease encoded in the expression system is a Streptococcus pyogenes Cas9 (SpCas9), Staphylococcus aureus Cas9 (SaCas9), a Francisella novicida Cas12a, Neisseria meningitidis Cas9 (Nme2Cas9), or a combination thereof.
17. In some embodiments, a cell or population of cells is provided comprising the expression system described herein.
18. In some embodiments, a method is provided comprising editing an endogenous MYH7 gene in at least one cell to reduce expression of a mutant MYH7 protein correlated with a cardiomyopathy.
19. In some embodiments, the endogenous MYH7 gene is heterozygous with a mutation in one MYH7 allele.
20. In some embodiments, the mutation in one MYH7 allele is a dominant mutation.
21. In some embodiments, the mutation in one MYH7 allele causes expression of the mutant MYH7 protein.
22. In some embodiments, editing the endogenous MYH7 gene eliminates expression of the mutant MYH7 protein.
23. In some embodiments, editing the endogenous MYH7 gene comprises CRISPR modification of one allele of the endogenous MYH7 gene.
24. In some embodiments, editing the endogenous MYH7 gene comprises nuclease cleavage of the endogenous MYH7 gene within at least one site recognized by at least one guide RNA.
25. In some embodiments, editing the endogenous MYH7 gene comprises guide RNA recognition of a MYH7 genomic site comprising a single nucleotide polymorphism (SNP).
26. In some embodiments, the SNP is selected from rs2069540, rs735711, rs7157716, rs2231126, or rs2069542.
27. In some embodiments, the guide RNA used in editing the endogenous MYH7 gene comprises an RNA sequence having at least 80%, at least 85%, at least 90%, at least 93%, at least 95%, at least 96%, at least 97%, or at least 98% sequence identity to any of SEQ ID NOs: 1-6 or 13-24, such as SEQ ID NOs: 14, 17, 18, 19, 22, 23, or 24.
28. In some embodiments, editing the endogenous MYH7 gene comprises contacting at least one cell with (a) a ribonucleoprotein complex comprising at least one nuclease and at least one guide RNA; or (b) an expression system comprising at least one expression cassette or expression vector comprising a promoter operably linked to a nucleic acid segment encoding at least one guide RNA, at least one nuclease, or a combination of at least one guide RNA and at least one nuclease, or mRNA of the guide RNA and/or nuclease.
29. In some embodiments, the at least one cell is a population of cells.
30. In some embodiments, the at least one cell is maintained in vitro in a culture medium.
31. In some embodiments, the at least one cell is allogeneic or autologous to a subject suspected of having the cardiomyopathy.
32. In some embodiments, the at least one cell is a cardiac cell, cardiac tissue, or a population of cardiac cells.
33. In some embodiments, the method further comprises confirming that expression of the mutant MYH7 protein correlated with a cardiomyopathy is reduced or eliminated in the at least one cell.
34. In some embodiments, the method further comprises administering the at least one cell with the reduced or eliminated mutant MYH7 protein expression to a subject.
35. In some embodiments, the at least one cell is in vivo within a subject, and the subject is suspected of having the cardiomyopathy.
36. In some embodiments, contacting at least one cell comprises (a) administering at least one guide RNA and a nuclease to the subject; or (b) administering to the subject an expression system comprising at least one expression cassette comprising a promoter operably linked to a nucleic acid segment encoding at least one guide RNA, at least one nuclease, or a combination of at least one guide RNA and at least one nuclease.
37. In some embodiments, a ribonucleoprotein complex comprising the at least one guide RNA and the nuclease is administered to the subject.
38. In some embodiments, a ribonucleoprotein complex comprising the at least one guide RNA and the nuclease is locally administered to the subject.
39. In some embodiments, a ribonucleoprotein complex comprising the at least one guide RNA and the nuclease is administered to cardiac cells, cardiac tissue, or a population of cardiac cells in the subject.
40. In some embodiments, a method is provided comprising administering the guide RNA described herein to a cell or to a subject.
41. In some embodiments, the guide RNA is administered to cardiac tissues or cardiac cells.
42. In some embodiments, a method is provided comprising administering the cell or the population of cells comprising the guide RNA to a subject.
43. In some embodiments, the cell or the population of cells comprising the guide RNA is administered to cardiac tissues or cardiac cells.
44. In some embodiments, a method is provided comprising administering the composition described herein to a cell or to a subject.
45. In some embodiments, the composition is administered to cardiac tissues or cardiac cells.
46. In some embodiments, a method is provided comprising administering the cell or population of cells comprising the composition to a subject.
47. In some embodiments, a method is provided comprising administering the ribonucleoprotein complex described herein to a cell or to a subject.
48. In some embodiments, the cell to which the ribonucleoprotein complex is administered is a cardiac cell or a cardiac tissue.
49. In some embodiments, a cell or population of cells is provided comprising the ribonucleoprotein complex described herein.
50. In some embodiments, a method is provided comprising administering the cell or the population of cells comprising the ribonucleoprotein complex to a subject.
51. In some embodiments, a method is provided comprising administering the expression system described herein to a cell or to a subject.
52. In some embodiments, the cell to which the expression system is administered is a cardiac cell or a cardiac tissue.
53. In some embodiments, a method is provided comprising administering the cell or the population of cells comprising the expression system to a subject.
54. In some embodiments, the cell or the population of cells comprising the expression system are administered to cardiac tissues of the subject.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 16th Edition, by M. Larranga, R Lewis Sr., and R Lewis, New York, N.Y., 2016.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described. As used herein, the term “in some embodiments” refers to embodiments of all aspects of the disclosure, unless the context clearly indicates otherwise.

The term “about” as used herein when referring to a measurable value such as an amount, a length, and the like, is meant to encompass variations of ±20% or ±10%, including ±5%, including ±1%, and including ±0.1% from the specified value.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

“Recombinant” as used herein to describe a nucleic acid molecule means a polynucleotide of genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “cell” refers to any type of cell. In some cases the cell is a cardiac cell. The cell can be in an organism or it can be maintained outside of an organism. The cell can be within a living organism and be in its normal (native) state. The term “cell” includes an individual cell or a group or population of cells. The cell(s) can be a prokaryotic, eukaryotic, or archaeon cell(s), such as a bacterial, archaeal, fungal, protist, plant, or animal cell(s). The cell(s) can be from or be within tissues, organs, and biopsies. The cell(s) can be a recombinant cell(s), a cell(s) from a cell line cultured in vitro. The cell(s) can include cellular fragments, cell components, or organelles comprising nucleic acids. In some cases, the cell(s) are human cells. The term cell(s) also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transfection, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected RNA or a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, including at least 8 to 10 amino acids, including at least 15 to 20 amino acids from a polypeptide encoded by the nucleic acid sequence. The RNA sequence or a portion thereof contains a nucleotide sequence of at least 3 to 5 nucleotides, including at least 8 to 10 nucleotides, and including at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

“Expression” refers to detectable production of a gene product by a cell. The gene product may be a transcription product (i.e., RNA), which may be referred to as “gene expression”, or the gene product may be a translation product of the transcription product (i.e., a protein), depending on the context.

“Purified polynucleotide” refers to a polynucleotide of interest or fragment thereof which is essentially free, e.g., contains less than about 50%, including less than about 70%, and including less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, including 80%-85%, including 90-95% of the sample. Techniques for purifying polynucleotides and polypeptides of interest are well-known in the art and include, for example, ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

The term define “transduction” refers to the introduction of foreign nucleic acid to a cell through a replication-incompetent viral vector.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Mammalian cell” refers to any cell derived from a mammalian subject suitable for transfection with an engineered vector system comprising an expression system described herein. The cell may be xenogeneic, autologous, or allogeneic. The cell can be a primary cell obtained directly from a mammalian subject. The cell may also be a cell derived from the culture and expansion of a cell obtained from a mammalian subject. Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, including at least about 8 nucleotides, including at least about 10-12 nucleotides, and including at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks) or minimal Cas target DNA or RNA binding activity. A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

In general, “a CRISPR system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR adaption system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays.

In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologs thereof, or modified versions thereof.

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In one embodiment, the protospacer is a defined synthetic DNA. In some embodiments, the defined synthetic DNA is at least 3, 5, 10, 20, 30, 40, or 50 nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. BacterioL, 169:5429-5433 (1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol. Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002); and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g., Jansen et al, Mol. Microbiol., 43:1565-1575 (2002); and Mojica et al, (2005)) including, but not limited to Aeropyrum, Pyrobaculum, Sulfolobus, Archaeoglobus, Halocarcula, Methanobacteriumn, Methanococcus, Methanosarcina, Methanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

The term “carrier” as used herein refers to a vehicle, matrix, or delivery agent that facilitates the transport, stability, or cellular uptake of one or more active agents, such as a guide RNA, a nuclease, a ribonucleoprotein complex, a nucleic acid, or a combination thereof. A carrier may be a biological carrier, a synthetic carrier, or a combination thereof. Examples of carriers include, but are not limited to, lipid nanoparticles (LNPs), virus-like particles (VLPs), adeno-associated virus (AAV) vectors, liposomes, polymeric nanoparticles, exosomes, microvesicles, and pharmaceutically acceptable excipients. A carrier does not include water or saline alone, unless combined with one or more additional components that facilitate delivery.

The term “sequence identity” as used herein refers to the percentage of nucleotide positions in an alignment of two sequences at which the nucleotides are the same. Sequence identity is determined by comparing two optimally aligned sequences over a comparison window, wherein the portion of the sequence in the comparison window may comprise additions or deletions (i.e., gaps) as compared to the reference sequence for optimal alignment of the two sequences. The percentage is calculated by determining the number of positions at which the identical nucleotide occurs in both sequences to yield the number of matched positions, dividing the number of matched positions by the total number of positions in the comparison window, and multiplying the result by 100 to yield the percentage of sequence identity. Sequence identity may be determined using standard algorithms and software known in the art, such as BLAST (Basic Local Alignment Search Tool), FASTA, or ClustalW.

The term “single nucleotide polymorphism” or “SNP” as used herein refers to a variation in a single nucleotide that occurs at a specific position in the genome, where each variation is present in at least 1% of the population. In the context of the present disclosure, the SNPs targeted by the guide RNAs are located within protein-coding exons of the MYH7 gene and include rs2069540, rs735711, rs7157716, rs2231126, and rs2069542. Each SNP has a reference allele and an alternate allele. A “heterozygous SNP” refers to a SNP position at which an individual carries two different alleles, one on each homologous chromosome.

The term “allele” as used herein refers to one of two or more alternative forms of a gene or a specific genomic locus that occupy the same position on homologous chromosomes. In a diploid organism, each individual carries two alleles for each autosomal locus, one inherited from each parent. As used herein, a “reference allele” refers to the allele corresponding to the nucleotide present in the reference human genome assembly at a given SNP position, and an “alternate allele” refers to a nucleotide at the same position that differs from the reference allele.

The term “in cis” as used herein refers to the arrangement of two or more genetic elements (e.g., a SNP and a disease-causing mutation) on the same chromosome, i.e., on the same physical DNA molecule. Two genetic elements that are in cis are inherited together on the same allele. This is distinguished from “in trans,” in which two genetic elements are located on different homologous chromosomes.

The term “protein-coding exon” as used herein refers to a segment of a gene that is both transcribed into mRNA and translated into a polypeptide. Protein-coding exons exclude untranslated regions (UTRs) that are transcribed but not translated. In the context of the MYH7 gene, protein-coding exons are those exons whose sequences encode portions of the beta-myosin heavy chain protein (SEQ ID NO: 7).

The term “heterozygous” as used herein refers to a genetic state in which the two alleles at a particular locus on homologous chromosomes are different from each other. A cell or subject that is heterozygous for a mutation in the MYH7 gene carries one allele with the mutation and one allele without the mutation (i.e., the wild-type allele) at the same locus.

The term “dominant mutation” as used herein refers to a mutation in one allele of a gene that produces a phenotypic effect even when the other allele is wild type. In the context of MYH7, a dominant mutation includes a dominant-negative mutation, in which the protein product of the mutant allele interferes with the function of the protein product of the wild-type allele when both are expressed.

The term “premature termination codon” as used herein refers to a stop codon (UAA, UAG, or UGA) that occurs in an mRNA transcript upstream of the normal stop codon of the coding sequence. A premature termination codon may arise as a result of a frameshift caused by an insertion or deletion (indel) in the genomic DNA, or as a result of a point mutation. A premature termination codon in an mRNA transcript may trigger degradation of that transcript by the nonsense-mediated decay (NMD) pathway.

The term “nonsense-mediated decay” or “NMD” as used herein refers to a cellular mRNA surveillance pathway that identifies and degrades mRNA transcripts containing premature termination codons. NMD reduces or eliminates protein expression from transcripts bearing premature termination codons. In the context of the present disclosure, NMD is instigated when a CRISPR-directed nuclease introduces an indel in a protein-coding exon of the MYH7 gene, generating a frameshift that produces a premature termination codon in the resulting mRNA transcript.

The term “insertion or deletion” or “indel” as used herein refers to a mutation in a nucleic acid sequence involving the addition of one or more nucleotides (insertion) or the removal of one or more nucleotides (deletion) at a specific position in the sequence. In the context of CRISPR-based gene editing, an indel is generated at or near the site of nuclease cleavage as a result of repair by the non-homologous end joining (NHEJ) pathway or other endogenous DNA repair mechanisms.

The term “lipid nanoparticle” or “LNP” as used herein refers to a particle having at least one dimension on the order of nanometers (e.g., 1-1,000 nm) that comprises one or more lipid components. LNPs can encapsulate or associate with nucleic acids, proteins, ribonucleoprotein complexes, or combinations thereof for delivery to cells. LNPs may include ionizable lipids, helper lipids, cholesterol, PEG-lipids, or combinations thereof.

The term “virus-like particle” or “VLP” as used herein refers to a non-replicative particle that resembles a virus in structure but lacks a viral genome capable of replication. VLPs may be derived from viral structural proteins and can be engineered to package and deliver cargo such as ribonucleoprotein complexes, proteins, nucleic acids, or combinations thereof to target cells. VLPs provide transient delivery of cargo without integrating into the host genome.

The term “adeno-associated virus” or “AAV” as used herein refers to a small, non-enveloped, replication-defective virus of the genus Dependoparvovirus. AAV vectors are derived from adeno-associated virus and engineered to carry a transgene flanked by AAV inverted terminal repeats (ITRs) in place of the viral coding sequences. AAV vectors can transduce both dividing and non-dividing cells and can provide long-term transgene expression. Multiple AAV serotypes exist (e.g., AAV1, AAV2, AAV5, AAV6, AAV8, AAV9, AAVrh10), each with different tissue tropism profiles.

The term “iPSC-derived cardiomyocyte” as used herein refers to a cardiomyocyte that has been differentiated in vitro from an induced pluripotent stem cell (iPSC). An iPSC is a pluripotent stem cell generated by reprogramming a somatic cell through the introduction of reprogramming factors. iPSC-derived cardiomyocytes exhibit properties of cardiac muscle cells, including expression of cardiac-specific markers and spontaneous contractile activity.

The term “cardiomyocyte” as used herein refers to a cardiac muscle cell. Cardiomyocytes are the cells responsible for generating contractile force in the heart. As used herein, the term includes primary cardiomyocytes isolated from cardiac tissue, cardiomyocytes differentiated from stem cells or progenitor cells (including iPSC-derived cardiomyocytes), and cardiomyocytes maintained in cell culture.

The term “cardiac tissue” as used herein refers to tissue of the heart comprising cardiomyocytes and associated cells, including but not limited to fibroblasts, endothelial cells, and smooth muscle cells. Cardiac tissue includes myocardial tissue of the left ventricle, right ventricle, atria, and interventricular septum.

The term “cardiac-specific promoter” as used herein refers to a promoter that directs transcription of an operably linked nucleic acid sequence preferentially or selectively in cardiac cells relative to non-cardiac cells. Examples of cardiac-specific promoters include, but are not limited to, the cardiac troponin T (cTnT) promoter, the alpha-myosin heavy chain (alpha-MHC) promoter, the myosin light chain 2v (MLC2v) promoter, and the cardiac troponin I (cTnI) promoter.

The term “adenosine base editor” or “ABE” as used herein refers to a fusion protein comprising a catalytically impaired Cas nuclease (e.g., a Cas9 nickase containing a D10A mutation) and an adenosine deaminase domain. An ABE converts adenine (A) to inosine (I) at a target position in genomic DNA, which is read as guanine (G) by cellular polymerases, resulting in an A-to-G (or T-to-C on the complementary strand) base conversion. Unlike a standard Cas9 nuclease, an ABE does not generate a double-strand break at the target site. As used herein, the term “Cas nuclease” encompasses an ABE comprising a Cas9 nickase domain when used in the context of directing a guide RNA to a target genomic site.

The term “Cas9 nickase” or “Cas9n” as used herein refers to a variant of a Cas9 nuclease in which one of the two nuclease domains (RuvC or HNH) has been inactivated by mutation, such that the protein cleaves only one strand of double-stranded DNA (i.e., generates a nick rather than a double-strand break). For example, a D10A mutation in SpCas9 inactivates the RuvC domain, producing a nickase that cleaves only the strand complementary to the guide RNA.

The term “cardiomyopathy” as used herein refers to a disease of the heart muscle that affects the structure or function of the myocardium. Cardiomyopathy includes, but is not limited to, hypertrophic cardiomyopathy (HCM), dilated cardiomyopathy (DCM), restrictive cardiomyopathy (RCM), and arrhythmogenic cardiomyopathy. As used herein, cardiomyopathy includes cardiomyopathies caused by or correlated with one or more mutations in the MYH7 gene.

The term “Neisseria meningitidis Cas9” or “Nme2Cas9” as used herein refers to a Cas9 ortholog derived from Neisseria meningitidis. Nme2Cas9 recognizes a PAM sequence of N4CC. Nme2Cas9 is smaller than SpCas9, which may facilitate packaging in size-constrained delivery vehicles such as AAV vectors.

The term “Staphylococcus aureus Cas9” or “SaCas9” as used herein refers to a Cas9 ortholog derived from Staphylococcus aureus. SaCas9 recognizes a PAM sequence of NNGRRT (where N is any nucleotide and R is a purine). SaCas9 is smaller than SpCas9 and can be packaged together with a guide RNA expression cassette in a single AAV vector.

The term “administering” as used herein refers to providing or delivering one or more agents (e.g., a guide RNA, a Cas nuclease, a ribonucleoprotein complex, a composition, a cell, or a population of cells) to a subject by any route. Routes of administration include, but are not limited to, intravenous, intracardiac, intramyocardial, intrapericardial, intracoronary, subcutaneous, intramuscular, intraperitoneal, and local injection. Administering also includes delivery via a catheter, implantable device, or other delivery apparatus.

The term “treating” or “treatment” as used herein refers to an intervention performed with the intention of preventing, slowing, halting, or reversing the progression of a disease or condition in a subject. In the context of the present disclosure, treating a subject having a heterozygous dominant mutation in the MYH7 gene correlated with cardiomyopathy includes reducing or eliminating expression of a mutant MYH7 protein from the allele carrying the mutation in cardiac cells of the subject. Treatment does not require that the disease or condition be completely cured or eliminated.

The term “mutant MYH7 protein” as used herein refers to a beta-myosin heavy chain protein encoded by an MYH7 allele that carries one or more mutations correlated with cardiomyopathy. The mutant MYH7 protein may differ from the wild-type MYH7 protein (SEQ ID NO: 7) by one or more amino acid substitutions, deletions, insertions, or truncations. The mutant MYH7 protein may exhibit altered function relative to the wild-type protein, including dominant-negative interference with sarcomeric assembly or contractile function.

The term “wild-type allele” as used herein refers to the allele of a gene that encodes the normal, non-disease-associated form of the gene product. In the context of MYH7, the wild-type allele encodes a beta-myosin heavy chain protein that supports normal cardiac function. A wild-type MYH7 allele does not carry a mutation correlated with cardiomyopathy.

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A composition comprising: (a) a carrier; (b) a guide RNA comprising an RNA sequence having at least 95% sequence identity to a sequence selected from the group consisting of SEQ ID NOs: 1-6, 13-24 and 25; and (c) a Cas nuclease, wherein the guide RNA directs the Cas nuclease to a genomic site within a protein-coding exon of a human MYH7 gene comprising a heterozygous single nucleotide polymorphism (SNP).

2. The composition of claim 1, wherein the guide RNA comprises an RNA sequence selected from the group consisting of SEQ ID NOs: 14, 17, 18, 19, 22, 23, 24 and 25.

3. The composition of claim 1, wherein the Cas nuclease is selected from the group consisting of Streptococcus pyogenes Cas9 (SpCas9), Staphylococcus aureus Cas9 (SaCas9), Francisella novicida Cas12a (FnCas12a), Neisseria meningitidis Cas9 (Nme2Cas9), and combinations thereof.

4. The composition of claim 1, wherein the guide RNA and the Cas nuclease are complexed as a ribonucleoprotein (RNP).

5. The composition of claim 1, wherein the carrier comprises a lipid nanoparticle, a virus-like particle (VLP), or an adeno-associated virus (AAV) vector.

6. The composition of claim 1, further comprising a second guide RNA comprising an RNA sequence having at least 95% sequence identity to a sequence selected from the group consisting of SEQ ID NOs: 1-6, 13-24 and 25, wherein the second guide RNA targets a different SNP allele than the first guide RNA.

7. The composition of claim 1, wherein the composition comprises an expression vector comprising a first promoter operably linked to a nucleic acid encoding the guide RNA and a second promoter operably linked to a nucleic acid encoding the Cas nuclease.

8. The composition of claim 7, wherein at least one of the first promoter and the second promoter is a cardiac-specific promoter.

9. A method of editing an endogenous MYH7 gene in a cell, the method comprising: contacting the cell with (i) a guide RNA comprising an RNA sequence having at least 95% sequence identity to a sequence selected from the group consisting of SEQ ID NOs: 1-6, 13-24 and 25, and (ii) a Cas nuclease, wherein the guide RNA directs the Cas nuclease to cleave one allele of the MYH7 gene at a genomic site comprising a heterozygous single nucleotide polymorphism (SNP), thereby introducing an insertion or deletion that generates a premature termination codon in the cleaved allele, and wherein expression of a mutant MYH7 protein encoded by the cleaved allele is reduced relative to expression prior to the contacting.

10. The method of claim 9, wherein the premature termination codon instigates nonsense-mediated decay of an mRNA transcript from the cleaved allele.

11. The method of claim 9, wherein the cell is heterozygous for a dominant mutation in the MYH7 gene, and the SNP is in cis with the dominant mutation on the same allele.

12. The method of claim 9, wherein the guide RNA and the Cas nuclease are delivered to the cell as a ribonucleoprotein (RNP) complex.

13. The method of claim 9, wherein the guide RNA and the Cas nuclease are delivered to the cell via a virus-like particle (VLP).

14. The method of claim 9, wherein the guide RNA comprises an RNA sequence selected from the group consisting of SEQ ID NOs: 14, 17, 18, 19, 22, 23, 24 and 25.

15. The method of claim 9, wherein the cell is an iPSC-derived cardiomyocyte or a cardiomyocyte.

16. The method of claim 9, wherein the Cas nuclease is SpCas9 or an adenosine base editor (ABE) comprising a Cas9 nickase domain.

17. A method of treating a subject having a heterozygous dominant mutation in the MYH7 gene correlated with cardiomyopathy, the method comprising administering to the subject (i) a guide RNA comprising an RNA sequence having at least 95% sequence identity to a sequence selected from the group consisting of SEQ ID NOs: 1-6, 13-24 and 25, and (ii) a Cas nuclease, wherein the guide RNA directs the Cas nuclease to a genomic site within a protein-coding exon of the MYH7 gene comprising a heterozygous single nucleotide polymorphism (SNP), wherein the SNP is in cis with the dominant mutation, and wherein the administering reduces expression of a mutant MYH7 protein from the allele carrying the dominant mutation in cardiac cells of the subject.

18. The method of claim 17, wherein the guide RNA and the Cas nuclease are administered as a ribonucleoprotein complex within a virus-like particle (VLP) or a lipid nanoparticle (LNP).

19. The method of claim 17, wherein the guide RNA comprises an RNA sequence selected from the group consisting of SEQ ID NOs: 14, 17, 18, 19, 22, 23, 24 and 25.

20. The method of claim 17, wherein the administering comprises delivering the guide RNA and the Cas nuclease to cardiac tissue of the subject, and wherein expression from a wild-type MYH7 allele in the cardiac tissue is maintained.


SEQUENCE LISTING (25 sequences)

SEQ ID NO: 1 - DNA, 20 bp, synthetic construct
  source 1..20: mol_type = other DNA
caccttgccatactcagtct

SEQ ID NO: 2 - DNA, 20 bp, synthetic construct
  source 1..20: mol_type = other DNA
caccttgccatactcggtct

SEQ ID NO: 3 - DNA, 20 bp, synthetic construct
  source 1..20: mol_type = other DNA
gaagttcaaactgaagcagc

SEQ ID NO: 4 - DNA, 20 bp, synthetic construct
  source 1..20: mol_type = other DNA
gaagttcaagctgaagcagc

SEQ ID NO: 5 - DNA, 20 bp, synthetic construct
  source 1..20: mol_type = other DNA
tgaagttcaaactgaagcag

SEQ ID NO: 6 - DNA, 20 bp, synthetic construct
  source 1..20: mol_type = other DNA
tgaagttcaagctgaagcag

SEQ ID NO: 7 - protein, 1935 aa, Homo sapiens
MGDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 8 - DNA, 6027 bp, Homo sapiens
  source 1..6027: mol_type = other DNA
acagctctgtcctgctctgtgtctttccctgctgctctcaggtcccctgcaggccttggcccctttcctcatctgtagacacacttgagtagcccaggcacagccatgggagattcggagatggcagtctttggggctgccgccccctacctgcgcaagtcagagaaggagcggctagaagcgcagaccaggccttttgacctcaagaaggatgtcttcgtgcctgatgacaaacaggagtttgtcaaggccaagatcgtgtctcgagagggtggcaaagtcactgccgagaccgagtatggcaagacagtgaccgtgaaggaggaccaggtgatgcagcagaacccacccaagttcgacaaaatcgaggacatggccatgctgaccttcctgcatgagcccgcggtgctctacaacctcaaggatcgctacggctcctggatgatctacacctactcgggcctcttctgtgtcaccgtcaacccttacaagtggctgccggtgtacactcctgaggtggtggctgcctaccggggcaagaagaggagcgaggccccgccccacatcttctccatctccgacaacgcctatcagtacatgctgacagacagagaaaaccagtccatcctgatcaccggagaatccggagcagggaagacagtcaacaccaagagggtcatccagtactttgctgttattgcagccattggggaccgcagcaagaaggaccagagcccgggcaagggcaccctggaggaccagatcatccaggccaaccctgctctggaggcctttggcaatgccaagaccgtccggaacgacaactcctcccgcttcgggaaattcattcgaattcattttggggcaacaggaaagttggcatctgcagacatagagacctatcttctggaaaaatccagagttattttccagctgaaagcagagagagattatcacattttctaccaaatcctgtctaacaaaaagcctgagctgctggacatgctgctgatcaccaacaacccctacgattatgcattcatctcccaaggagagaccaccgtggcctccattgatgacgctgaggagctcatggccactgataacgcttttgatgtgctgggcttcacttcagaggagaaaaactccatgtataagctgacaggcgccatcatgcactttggaaacatgaagttcaagctgaagcagcgggaggagcaggcggagccagacggcactgaagaggctgacaagtctgcctacctcatggggctgaactcagccgacctgctcaaggggctgtgccaccctcgggtgaaagtgggcaatgagtacgtcaccaaggggcagaatgtccagcaggtgatatatgccactggggcactggccaaggcagtgtatgagaggatgttcaactggatggtgacgcgcatcaatgccaccctggagaccaagcagccacgccagtacttcataggagtcctggacatcgctggcttcgagatcttcgatttcaacagctttgagcagctctgcatcaacttcaccaacgagaagctgcagcagttcttcaaccaccacatgtttgtgctggagcaggaggagtacaagaaggagggcatcgagtggacattcattgactttggcatggacctgcaggcctgcattgacctcatcgagaagcccatgggcatcatgtccatcctggaagaggagtgcatgttccccaaggccaccgacatgaccttcaaggccaagctgtttgacaaccacctgggcaaatccgccaacttccagaagccacgcaatatcaaggggaagcctgaagcccacttctccctgatccactatgccggcatcgtggactacaacatcattggctggctgcagaagaacaaggatcctctcaatgagactgtcgtgggcttgtatcagaagtcttccctcaagctgctcagcaccctgtttgccaactatgctggggctgatgcgcctattgagaagggcaaaggcaaggccaagaaaggctcgtcctttcagactgtgtcagctctgcacagggaaaatctgaacaagctgatgaccaacttgcgctccacccatccccactttgtacgttgtatcatccctaatgagacaaagtctccaggggtgatggacaaccccctggtcatgcaccagctgcgctgcaatggtgtgctggagggcatccgcatctgcaggaaaggcttccccaaccgcatcctctacggggacttccggcagaggtatcgcatcctgaacccagcggccatccctgagggacagttcattgatagcaggaagggggcagagaagctgctcagctccctggacattgatcacaaccagtacaagtttggccacaccaaggtgttcttcaaggccgggctgctggggctgctggaggaaatgagggacgagaggctgagccgcatcatcacgcgtatccaggcccagtcccgaggtgtgctcgccagaatggagtacaaaaagctgctggaacgtagagactccctgctggtaatccagtggaacattcgggccttcatgggggtcaagaattggccctggatgaagctctacttcaagatcaagccgctgctgaagagtgcagaaagagagaaggagatggcctccatgaaggaggagttcacacgcctcaaagaggcgctagagaagtccgaggctcgccgcaaggagctggaggagaagatggtgtccctgctgcaggagaagaatgacctgcagctccaagtgcaggcggaacaagacaacctggcagatgctgaggagcgctgtgatcagctgatcaaaaacaagattcagctggaggccaaggtgaaggagatgaacgagaggctggaggatgaggaggagatgaatgctgagctcactgccaagaagcgcaagctggaagatgagtgctcagagctcaaaagggacatcgatgatctggagctgacactggccaaagtggagaaggagaaacacgcaacagagaacaaggtgaaaaacctgacagaggagatggctgggctggatgagatcattgccaagctgaccaaggagaagaaagctctgcaagaggcccaccaacaggctctggatgaccttcaggccgaggaggacaaggtcaacaccctgactaaggccaaagtcaagctggagcagcaagtggatgatctggaaggatccctggagcaagagaagaaggtgcgcatggacctggagcgagcgaagcggaagctggagggcgacctgaagctgacccaggagagcatcatggacctggagaatgacaagcagcagctggatgagcggctgaaaaaaaaagactttgagctgaatgctctcaacgcaaggattgaggatgaacaggccctcggcagccagctgcagaagaagctcaaggagcttcaggcacgcatcgaggagctggaggaggagctggaggccgagcgcaccgccagggctaaggtggagaagctgcgctcagacctgtctcgggagctggaggagatcagcgagcggctggaagaggccggcggggccacgtccgtgcagatcgagatgaacaagaagcgcgaggccgagttccagaagatgcggcgggacctggaggaggccacgctgcagcacgaggccactgccgcggccctgcgcaagaagcacgccgacagcgtggccgagctgggcgagcagatcgacaacctgcagcgggtgaagcagaagctggagaaggagaagagcgagttcaagctggagctggatgacgtcacctccaacatggagcagatcatcaaggccaaggctaacctggagaagatgtgccggaccttggaagaccagatgaatgagcaccggagcaaggcggaggagacccagcgttctgtcaacgacctcaccagccagcgggccaagttgcaaaccgagaatggtgagctgtcccggcagctggatgagaaggaggcactgatctcccagctgacccgaggcaagctcacctacacccagcagctggaggacctcaagaggcagctggaggaggaggttaaggcgaagaacgccctggcccacgcactgcagtcggcccggcatgactgcgacctgctgcgggagcagtacgaggaggagacggaggccaaggccgagctgcagcgcgtcctttccaaggccaactcggaggtggcccagtggaggaccaagtatgagacggacgccattcagcggactgaggagctcgaggaggccaagaagaagctggcccagcggctgcaggaagctgaggaggccgtggaggctgttaatgccaagtgctcctcgctggagaagaccaagcaccggctacagaatgagatcgaggacttgatggtggacgtagagcgctccaatgctgctgctgcagccctggacaagaagcagaggaacttcgacaagatcctggccgagtggaagcagaagtatgaggagtcgcagtcggagctggagtcctcgcagaaggaggctcgctccctcagcacagagctcttcaaactcaagaacgcctatgaggagtccctggaacatctggagaccttcaagcgggagaacaaaaacctgcaggaggagatctccgacttgactgagcagttgggttccagcggaaagactatccatgagctggagaaggtccgaaagcagctggaggccgagaagatggagctgcagtcagccctggaggaggccgaggcctccctggagcacgaggagggcaagatcctccgggcccagctggagttcaaccagatcaaggcagagatcgagcggaagctggcagagaaggacgaggagatggaacaggccaagcgcaaccacctgcgggtggtggactcgctgcagacctccctggacgcagagacacgcagccgcaacgaggccctgagggtgaagaagaagatggaaggagacctcaatgagatggagatccagctcagccacgccaaccgcatggccgccgaggcccagaagcaagtcaagagcctccagagcttgttgaaggacacccagattcagctggacgatgcagtccgtgccaacgacgacctgaaggagaacatcgccatcgtggagcggcgcaacaacctgctgcaggctgagctggaggagttgcgtgccgtggtggagcagacagagcggtcccggaagctggcggagcaggagctgattgagactagtgagcgggtgcagctgctgcattcccagaacaccagcctcatcaaccagaagaagaagatggatgctgacctgtcccagctccagactgaagtggaggaggcagtgcaggagtgcaggaatgctgaggagaaggccaagaaggccatcacggatgccgccatgatggcagaggagctgaagaaggagcaggacaccagcgcccacctggagcgcatgaagaagaacatggaacagaccattaaggacctgcagcaccggctggacgaagccgagcagatcgccctcaagggcggcaagaagcagctgcagaagctggaagcgcgggtgcgggagctggagaatgagctggaggccgagcagaagcgcaacgcagagtcggtgaagggcatgaggaagagcgagcggcgcatcaaggagctcacctaccagacggaggaggacaggaaaaacctgctgcggctgcaggacctggtagacaagctgcagctaaaggtcaaggcctacaagcgccaggccgaggaggcggaggagcaagccaacaccaacctgtccaagttccgcaaggtgcagcacgagctggatgaggcagaggagcgggcggacatcgccgagtcccaggtcaacaagctgcgggccaagagccgtgacattggcacgaagggcttgaatgaggagtagctttgccacatcttgatctgctcagccctggaggtgccagcaaagccccatgctggagcctgtgtaacagctccttgggaggaagcagaataaagcaattttccttgaagccga

SEQ ID NO: 9 - protein, 1368 aa, Streptococcus pyogenes [sequence omitted]

SEQ ID NO: 10 - DNA, 4101 bp, Streptococcus pyogenes
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 11 - protein, 1307 aa, Francisella sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 12 - DNA, 3921 bp, Francisella sp.
  source 1..3921: mol_type = other DNA
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgcctccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgcgaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 13 - DNA, 20 bp, synthetic construct
  source 1..20: mol_type = other DNA
ctcccaccttcagtgccgtc

SEQ ID NO: 14 - DNA, 20 bp, synthetic construct
  source 1..20: mol_type = other DNA
ctcccaccttcagtgccatc

SEQ ID NO: 15 - DNA, 20 bp, synthetic construct
  source 1..20: mol_type = other DNA
gcggagccagacggcactga

SEQ ID NO: 16 - DNA, 19 bp, synthetic construct
  source 1..19: mol_type = other DNA
gcggagccagatggcactg

SEQ ID NO: 17 - DNA, 20 bp, synthetic construct
  source 1..20: mol_type = other DNA
gtcagggaccactcacgaag

SEQ ID NO: 18 - DNA, 20 bp, synthetic construct
  source 1..20: mol_type = other DNA
gtcagggaccactcacaaag

SEQ ID NO: 19 - DNA, 19 bp, synthetic construct
  source 1..19: mol_type = other DNA
tcagggaccactcacgaag

SEQ ID NO: 20 - DNA, 20 bp, synthetic construct
  source 1..20: mol_type = other DNA
tcagggaccactcacaaagc

SEQ ID NO: 21 - DNA, 20 bp, synthetic construct
  source 1..20: mol_type = other DNA
gggaccactcacgaagcggg

SEQ ID NO: 22 - DNA, 20 bp, synthetic construct
  source 1..20: mol_type = other DNA
gggaccactcacaaagcggg

SEQ ID NO: 23 - DNA, 20 bp, synthetic construct
  source 1..20: mol_type = other DNA
aactcctcccgcttcgtgag

SEQ ID NO: 24 - DNA, 20 bp, synthetic construct
  source 1..20: mol_type = other DNA
aactcctcccgctttgtgag

SEQ ID NO: 25 - DNA, 20 bp, synthetic construct
  source 1..20: mol_type = other DNA
gacacccaccttgccatact